(19) United States
(12) Patent Application Publication
(10) Pub. No.: US 2025/0339022 A1
(43) Pub. Date: Nov. 6, 2025

(54) MEDICAL USE SOFT ROBOTS

(71) Applicant: Vine Medical, Inc., Goleta, CA (US)

(72) Inventors: David HAGGERTY, Goleta (CA); Elliot HAWKES, Goleta, CA (US); James CAZZOLI, Goleta, CA (US); David DROVER, Stanford, CA (US); Jose ZEVALLOS, Pittsburgh, PA (US)

(21) Appl. No.: 19/014,154

(22) Filed: Jan. 8, 2025

Related U.S. Application Data
(63) Continuation-in-part of application No. PCT/US2023/076294, filed on Oct. 6, 2023.
(60) Provisional application No. 63/413,832, filed on Oct. 6, 2022.
(60) Provisional application No. 63/437,893, filed on Jan. 9, 2023.
(60) Provisional application No. 63/582,035, filed on Sep. 12, 2023.
(60) Provisional application No. 63/619,237, filed on Jan. 9, 2024.

(51) Int. Cl. A61B 1/267; A61B 1/00; A61B 1/018; A61B 1/04; A61B 34/30

(52) U.S. Cl. CPC A61B 1/267; A61B 1/00045; A61B 1/00097; A61B 1/018; A61B 1/042; A61B 34/30; A61B 2034/303

(57) ABSTRACT

Provided herein are systems, methods, and platforms that enable tracheal intubation with improved ease and reduced risk of patient harm. These systems, methods, and platforms can include introduction devices and systems for tracheal intubation. These systems, methods, and platforms can include intubation device. The intubation devices can include robotic components.

### CROSS-REFERENCE

This application is a continuation-in-part of and claims the benefit of PCT Application No. PCT/US23/76294, which claims the benefit of U.S. Provisional Application No. 63/413,832 filed Oct. 6, 2022, U.S. Provisional Application No. 63/437,893 filed Jan. 9, 2023, and U.S. Provisional Application No. 63/582,035 filed Sep. 12, 2023; and this application also claims the benefit of U.S. Provisional Application No. 63/619,237 filed Jan. 9, 2024; all of which are hereby incorporated by reference in their entirety herein.

The subject matter of this patent application is related to that of PCT/US2023/076294 filed Oct. 6, 2023, which claims the benefit of U.S. Provisional Application No. 63/413,832 filed Oct. 6, 2022, U.S. Provisional Application No. 63/437,893 filed Jan. 9, 2023, and U.S. Provisional Application No. 63/582,035 filed Sep. 12, 2023; PCT/US2022/044162 filed Sep. 20, 2022, which claims priority to U.S. provisional applications Nos. 63/246,135 filed Sep. 20, 2021, 63/306,805 filed Feb. 4, 2022, and 63/399,597 filed Aug. 19, 2022; U.S. patent application Ser. No. 17/632,335 filed Feb. 2, 2022; PCT Application No. PCT/US2022/019257 filed Mar. 8, 2022; and PCT/US2020/043942 filed Jul. 29, 2020, which claims priority to U.S. provisional application No. 62/882,239 filed Aug. 2, 2019; all of which are incorporated herein in their entirety.

### BACKGROUND

The present disclosure generally relates to medical devices, systems, and methods, and in particular those suitable for facilitating procedures such as endoscopy and tracheal intubation.

Endoscopy is a procedure used in medicine to look inside the body. The endoscopy procedure uses an endoscope to examine the interior of a hollow organ or cavity of the body. Unlike many other medical imaging techniques, endoscopes are inserted directly into the organ, which can require a high degree of practitioner expertise and involve risk of patient harm.

Tracheal intubation, usually simply referred to as intubation, is the placement of a flexible plastic tube into the trachea to maintain an open airway or to serve as a conduit through which to administer fluids. Intubation similarly can require a high degree of practitioner expertise and involve risk of patient harm.

Hence, improved devices, systems, and methods to facilitate such procedures, reducing the degree of practitioner expertise required and reducing the risk of patient harm, are desired.

### SUMMARY

An aspect of the present disclosure provides an exemplary tracheal intubation device, comprising an anterior tracheal access mechanism (ATAM) comprising: a posterior shim comprising a first tine and a second tine; and an anterior shim comprising: a first portion coupled to the first tine; a second portion coupled to the second tine; and a first bridge connecting the first portion and the second portion, wherein the first bridge is offset from the posterior shim.

In some embodiments, the device further comprises a primary vine robot that is shaped and sized to extend through a patient's larynx when fully actuated. In some embodiments, the first tine and the second tine extend in an actuation direction of the primary vine robot. In some embodiments, the ATAM is shaped and sized to lift the epiglottis of a patient anteriorly when the primary vine robot is fully actuated. In some embodiments, the ATAM is removably coupled to an everting end of the primary vine robot. In some embodiments, the ATAM is rotatably and removably coupled to the everting end of the vine robot. In some embodiments, the vine robot comprises a camera.

In some embodiments, the ATAM further comprises a first tine cover surrounding at least a distal portion of the first tine, a second tine cover surrounding at least a distal portion of the second tine, or both. In some embodiments, the first tine cover, the second tine cover, or both comprise a coating. In some embodiments, the first tine cover, the second tine cover, or both are formed of a material having a modulus of elasticity less than the modulus of elasticity of the material of the posterior shim.

In some embodiments, a distal portion of the first portion of the anterior shim, a distal portion of the second portion of the anterior shim, or both, comprise a rounded nub.

In some embodiments, the first portion of the anterior shim comprises a primary first segment and a secondary first segment noncolinear to the primary first segment; the second portion of the anterior shim comprises a primary second segment and a secondary second segment noncolinear to the primary second segment; or both.

In some embodiments, the anterior shim further comprises a second bridge connecting the first portion and the second portion at a point proximal to the first bridge. In some embodiments, the anterior shim further comprises a cantilever extending distally from the second bridge. In some embodiments, the cantilever comprises a first cantilever portion and a second cantilever portion having a thickness less than a thickness of the first cantilever portion.

In some embodiments, the ATAM further comprises a webbing extending from at least two of the first portion, the second portion, and the first bridge.

In some embodiments, the first tine, the second tine, or both comprise a first flexible portion and a second flexible portion coupled to the distal terminus and the proximal terminus of the first flexible portion. In some embodiments, a mesial portion of the first flexible portion and a mesial portion of the second flexible portion are biased to separate. In some embodiments, the posterior shim further comprises an inflatable chamber between the first flexible portion and the second flexible portion of the first tine, the second tine, or both. In some embodiments, the posterior shim further comprises an inflatable chamber coupled to the first tine, the second tine, or both.

Another aspect of the present disclosure provides an exemplary tracheal intubation system comprising any of the tracheal intubation device described herein and an introducer device configured to accept said tracheal intubation device and to eject at least a portion of the tracheal intubation device into the mouth of the patient distal to the epiglottis.

In some embodiments, the introducer device comprises: an introducer comprising: a first slot sized to slidably receive the first tine of the posterior shim; a second slot sized to slidably receive the second tine of the posterior shim; and a channel between the first slot and the second slot sized to slidably receive the first bridge; and a plunger comprising: a first portion sized to slide within the first slot; a second portion sized to slide within the second slot; and an arcuate portion between the first portion and the second portion sized to slide within the channel of the introducer.

In some embodiments, the introducer system further comprises a disconnect configured to separate the introducer system from the vine robot.

In some embodiments, the introducer device comprises: a tubular body configured to receive the tracheal intubation device, wherein a distal end of the tubular body is angled with respect to a elongate axis of the tubular body; an ovular surface coplanar to the distal end and extending outwards from the elongate axis, wherein the ovular surface comprises a flap configured to, in a collapsed state, cover at least a portion of the distal end of the tubular body and, in an expanded state, extend perpendicular to the ovular surface. In some embodiments, an outer rim of the ovular surface has a greater thickness than the remainder of the ovular surface.

Another aspect of the present disclosure provides an exemplary method of intubating a patient comprising: (a) inserting the system herein into the mouth of the patient to the pharyngeal wall of the patient; (b) advancing the plunger distally toward the patient to eject the ATAM; (c) advancing the primary vine robot distally between the first bridge and the posterior shim of the ATAM; (d) disconnecting the ATAM from the primary vine robot; and (e) removing the ATAM and the introducer from the patient's mouth.

In some embodiments, the primary vine robot remains within the patient after step (e).

In some embodiments, the first bridge contacts the vallecula of the patient, the hyoepiglottic ligament of the patient, the glossoepiglottic folds of the patient, or any combination thereof after step (b). In some embodiments, during advancement, pressure applied by the first bridge lifts the epiglottis anteriorly. In some embodiments, the lifting of the epiglottitis anteriorly facilitates directing of the primary vine robot toward the trachea when being actuated.

Another aspect of the present disclosure provides an exemplary soft vine endoscopy robot, comprising a primary tube body having a sealed first end and a second end opposite the first end, wherein the first end is within a mesial portion of the primary tube body, and wherein the mesial portion comprises two or more stiffened portions, wherein adjacent stiffened portions are separated by a segment of the primary tube body. In some embodiments, the two or more stiffened portions are arranged from the first end to the second end. In some embodiments, the two or more stiffened portions are radially aligned about the primary tube body. In some embodiments, the two or more stiffened portions are radially arrayed about the primary tube body. In some embodiments, when inflated, the primary tube body has two non-colinear sections that intersect at one of the two or more stiffened portions. In some embodiments, the two or more stiffened portions have a thickness greater than the rest of the primary tube body. In some embodiments, the two or more stiffened portions have a modulus of elasticity less than the rest of the primary tube body. In some embodiments, the two or more stiffened portions comprises a stop coupled to the primary tube body.

In some embodiments, the robot comprises two or more stops, wherein the two or more stops are interconnected by a tendon. In some embodiments, the tendon has a constant length. In some embodiments, the tendon has an actuatable length. In some embodiments, the primary tube body comprises a plurality of segments separated from one another by the two or more stiffened portions. In some embodiments, the primary tube body, when inflated, is configured to have a plurality of segment lengths and angles between segments that match lengths and curvatures of a predefined bodily lumen.

In some embodiments, the robot further comprises an annular chassis having a cavity, wherein the primary tube body extends from the first end, through the cavity, around an outer surface of the annular chassis and to the mesial portion of the primary tube body.

In some embodiments, the chassis comprises one or more camera sensors. In some embodiments, the robot further comprises a camera body comprising: a proximal portion; a distal portion comprising one or more camera sensors, wherein the distal portion has an outer diameter greater than an inner diameter of the annular chassis; and a mesial portion between the distal portion and the proximal portion, wherein the mesial portion is sized to fit within the cavity of the annular chassis.

Another aspect of the present disclosure provides an exemplary method of forming a soft vine endoscopy robot, the method comprising: receiving an anatomical measurement comprising a length of a first portion, a length of a second portion, and an angle between the first portion and the second portion; stiffening a portion of a primary tube body based on the length of the first portion, the length of the second portion, the angle between the first portion and the second portion, or any combination thereof, wherein the primary tube body has a first end, a second end, and a channel therethrough; and inserting the first end through the channel at the second end. In some embodiments, stiffening the portion of the primary tube comprises heating the portion of the primary tube. In some embodiments, stiffening the portion of the primary tube comprises attaching a stop to the portion of the primary tube, wherein the stop is coupled to a tendon.

Another aspect of the present disclosure provides an exemplary a soft vine endoscopy robot, comprising: a primary tube body having a sealed first end, wherein the first end is within a mesial portion of the primary tube body; and a secondary tube body attached to an outer surface of the primary tube body, wherein the primary tube body and the secondary tube body are parallel or concentric. In some embodiments, inflating the tube body from the second end translates a distal end of the primary tube body distally relative to the sealed end. In some embodiments, inflating the secondary tube body bends the primary tube body about an attachment arc between the primary tube body and the secondary tube body.

Another aspect of the present disclosure provides an exemplary soft vine endoscopy robot, comprising: an endoscope connector comprising an endoscope fastener configured to fasten to an endoscope, a converging-diverging hole, and an inflation port; and a primary tube body having a first end coupled and sealed to an outer surface of the endoscope connector and a sealed second end that passes through the converging-diverging hole. In some embodiments, inflating the primary tube body through the inflation port extends a distal surface of the primary tube body away from the endoscope connector. In some embodiments, maintaining a set pressure within the primary tube body seals a mesial portion of the primary tube body against the converging-diverging hole.

Another aspect of the present disclosure provides an exemplary soft vine endoscopy robot, comprising: a primary tube body having a sealed first end, wherein the first end is within a mesial portion of the tube body; a retractor coupled to the first end of the primary tube body; and a motor configured to drive the retractor to collapse at least a portion of the primary tube body. In some embodiments, the retractor comprises a spool. In some embodiments, the retractor comprises a pulley.

Another aspect of the present disclosure provides an exemplary method of intubating a patient, the method comprising: inserting a vine robot in an unactuated position into the patient's mouth, wherein the vine robot comprises a primary tube body having a sealed first end and a second end, wherein the first end is within a mesial portion of the tube body; applying fluid pressure into the primary tube body to gradually evert and extend the primary tube body into the back of the laryngopharynx of the patient and into the trachea of the patient to provide a lumen from a mouthpiece to the trachea; and providing air or oxygen through the lumen into the trachea.

Another aspect of the present disclosure provides an exemplary method of accessing a cavity of a patient with a medical tool, the method comprising: inserting a vine robot in an unactuated position into the patient's mouth, wherein the vine robot comprises a primary tube body having a sealed first end and a second end, wherein the first end is within a mesial portion of the tube body; applying fluid pressure into the primary tube body to gradually evert and extend the primary tube body into the cavity of the patient to form a lumen into the cavity of the patient; and accessing the cavity of the patient through the lumen.

Another aspect of the present disclosure provides a tracheal intubation device comprising: an introducer; and a tracheal access mechanism (TAM) connected to the introducer, the TAM comprising: a base having an egress for passing an endotracheal tube (ETT) subassembly, a bridge extending from the base, and a flexible datum extending over the bridge. In some embodiments, the flexible datum is configured to conform to a person's hyoid bone.

In some embodiments, the TAM includes an ETT egress port offset and a datum having an upper esophageal sphincter (UES) self-centering width and molar avoidance geometry.

In some embodiments, the introducer includes a proximal connected member and at least two distal unconnected members connected to the proximal connected member. In some embodiments, the introducer includes a channel configured to enable passage of one or more tools, cameras, or catheters. In some embodiments, the introducer includes a visualization egress for a visualization device. In some embodiments, the visualization egress is a channel for securing an optical fiber or camera. In some embodiments, the introducer includes a gastric port configured to allow for an orogastric tube to be passed along the introducer. In some embodiments, the introducer includes a first member including the egress for passing the ETT subassembly and a second member including the gastric port. In some embodiments, the first member and the second member are unconnected members. In some embodiments, the TAM includes a TAM egress.

In some embodiments, the introducer includes a directionally-compliant portion along the length of the introducer configured to bend easily when in contact on a posterior surface and to be rigid in contact on an anterior surface. In some embodiments, the directionally-compliant portion includes series of groves configured to enhance bending in a preferential direction. In some embodiments, the directionally-compliant portion includes series of pillars configured to enhance bending in a preferential direction. In some embodiments, the directionally-compliant portion has a tunable stiffness. In some embodiments, the directionally-compliant portion includes a cover configured to resist or aid in bending. In some embodiments, the cover wraps around the introducer. In some embodiments, the directionally-compliant portion includes a first cover configured to add stiffness in a first direction to resist bending and a second cover configured to enhance elasticity in a second direction. In some embodiments, the directionally-compliant portion includes a filler material configured to add stiffness in one direction to resist bending.

Another aspect of the present disclosure provides a primary vine robot that is shaped and sized to extend through a patient's larynx when fully actuated.

Another aspect of the present disclosure provides a tracheal intubation device comprising: an introducer having a tracheal access mechanism (TAM) connected to a distal end of the introducer; a display holder configured to secure a visualization device; and a connector configured to releasably interface the display holder with a proximal end of the introducer, the connector including an ETT egress.

Another aspect of the present disclosure provides a removable ETT subassembly configured to pass through the ETT egress. Another aspect of the present disclosure provides a display configured to secure to the display holder and visualize a field of view from the TAM.

Another aspect of the present disclosure provides a tracheal intubation device comprising: a base including a hinge; a datum connected to a distal end of the base; a coupler connected to a proximal portion of the base and to the hinge; a lever secured to the base and configured to apply a force to the coupler, wherein the force is configured to move the datum.

Another aspect of the present disclosure provides a tracheal intubation device comprising: a pair of unconnected members; and a tracheal access mechanism (TAM) connected to a distal end of the pair of unconnected members.

Another aspect of the present disclosure provides a tracheal intubation device comprising: an introducer including a connected member and a pair of lateral rails configured to contact posterior tissue and to preserve a volume between the connected member; an anterior surface connected between the pair of lateral rails; and a datum connected to the pair of lateral rails; wherein the anterior surface is configured to the support the datum.

Another aspect of the present disclosure provides a tracheal intubation device comprising: a tracheal access mechanism (TAM); and an introducer connected to the TAM, the introducer including body, and a posterior valence mechanism or floating connectors configured to augment a curvature of the body and induce tension on a posterior side of the TAM. In some embodiments, the body of the introducer includes a notch configured to form a pivot point for bending.

Another aspect of the present disclosure provides a tracheal intubation device comprising: a connected introducer including a pair of unconnected members each having a centering pin on a distal end configured to self-center the device within an orifice of a person.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a side view of tines of a tracheal intubation device, per one or more embodiments herein.

FIG. 2 shows a perspective view of anterior and posterior tine layers of a tracheal intubation device (e.g., the device of FIG. 1), per one or more embodiments herein.

FIG. 3 shows a perspective view of overlapping shims of a tracheal intubation device (e.g., the device of FIG. 1), per one or more embodiments herein.

FIG. 4 shows a first perspective view of an active-pinning shim of a tracheal intubation device (e.g., the device of FIG. 1), per one or more embodiments herein.

FIG. 5 shows a second perspective view of an active-pinning shim of a tracheal intubation device (e.g., the device of FIG. 1), per one or more embodiments herein.

FIG. 6A shows an exemplary section view map of a human airway.

FIG. 6B shows a schematic of a vine robot having stiffened portions, per one or more embodiments herein.

FIG. 7A shows a schematic illustration of an exemplary first step in a point-controlled method of navigating a vine robot through a lumen and around an obstacle.

FIG. 7B shows a schematic illustration of an exemplary second step in a point-controlled method of navigating a vine robot through a lumen and around an obstacle.

FIG. 7C shows a schematic illustration of an exemplary third step in a point-controlled method of navigating a vine robot through a lumen and around an obstacle.

FIG. 7D shows a schematic illustration of an exemplary fourth step in a point-controlled method of navigating a vine robot through a lumen and around an obstacle.

FIG. 7E shows a schematic illustration of an exemplary fifth step in a point-controlled method of navigating a vine robot through a lumen and around an obstacle.

FIG. 7F shows a schematic illustration of an exemplary sixth step in a point-controlled method of navigating a vine robot through a lumen and around an obstacle.

FIG. 8 shows a section view of a tube body comprising a plurality of segments separated from one another by the two or more stiffened portions, per one or more embodiments herein.

FIG. 9 shows a section view illustration of a tube body comprising a first non-colinear section that intersects the tube body, per one or more embodiments herein.

FIG. 10A shows a side section view of a vine robot having an annular chassis, per one or more embodiments herein.

FIG. 10B shows a front section view illustration of a vine robot having an annular chassis, per one or more embodiments herein.

FIG. 11 shows a cross-sectioned side-view illustration of a vine robot having an annular chassis and a camera body, per one or more embodiments herein.

FIG. 12A shows a side-view illustration of an expanded vine robot comprising an endoscope connector, per one or more embodiments herein.

FIG. 12B shows a side-view illustration of a collapsed vine robot comprising an endoscope connector, per one or more embodiments herein.

FIG. 13 shows a cross-sectioned side-view illustration of a vine robot having a roller retraction mechanism, per one or more embodiments herein.

FIG. 14 shows a cross-sectioned side-view illustration of a vine robot having a reel retraction mechanism.

FIG. 15A shows a side-view illustration of a multi-walled vine robot, per one or more embodiments herein.

FIG. 15B shows a front-view illustration of an annular multi-walled vine robot, per one or more embodiments herein.

FIG. 15C shows a detailed side-view illustration of a reinforced multi-walled vine robot, per one or more embodiments herein.

FIG. 15D shows a front-view illustration of a radial multi-walled vine robot, per one or more embodiments herein.

FIG. 16A shows a front-left perspective view illustration of an exemplary orotracheal intubation introducer, per one or more embodiments herein.

FIG. 16B shows a side view illustration of an exemplary orotracheal intubation introducer, per one or more embodiments herein.

FIG. 16C shows a front view illustration of an exemplary orotracheal intubation introducer, per one or more embodiments herein.

FIG. 17A shows a first perspective view of an exemplary introducer with shims, per one or more embodiments herein.

FIG. 17B shows a second perspective view of an exemplary introducer with shims (e.g., the introducer of FIG. 17A), per one or more embodiments herein.

FIG. 17C shows a third perspective view n of an exemplary introducer with shims (e.g., the introducer of FIG. 17A), per one or more embodiments herein.

FIG. 18A shows a first perspective view of an exemplary introducer with a wedge, per one or more embodiments herein.

FIG. 18B shows a second perspective view of an exemplary introducer with a wedge (e.g., the introducer of FIG. 18A), per one or more embodiments herein.

FIG. 18C shows a third perspective view of an exemplary introducer with a wedge (e.g., the introducer of FIG. 18A), per one or more embodiments herein.

FIG. 18D shows a third perspective view of an exemplary introducer with a wedge (e.g., the introducer of FIG. 18A), per one or more embodiments herein.

FIG. 19A shows a first perspective view of an exemplary introducer with a vallecula stop, per one or more embodiments herein.

FIG. 19B shows a second perspective view of an exemplary introducer with a vallecula stop (e.g., the introducer of FIG. 19A), per one or more embodiments herein.

FIG. 19C shows a third perspective view of an exemplary introducer with a vallecula stop (e.g., the introducer of FIG. 19A), per one or more embodiments herein.

FIG. 20A shows a first side view illustration of an exemplary toroidal vine robot, per one or more embodiments herein.

FIG. 20B shows a second side view illustration of an exemplary toroidal vine robot (e.g., the vine robot of FIG. 20A), per one or more embodiments herein.

FIG. 20C shows a third perspective view illustration of an exemplary toroidal vine robot (e.g., the vine robot of FIG. 20A), per one or more embodiments herein.

FIG. 21A shows a first perspective view of a commercial Endotracheal Tube (ETT) sheathed in an exemplary toroidal vine robot, per one or more embodiments herein.

FIG. 21B shows a second perspective view of a commercial ETT sheathed in an exemplary toroidal vine robot (e.g., the ETT of FIG. 21A), per one or more embodiments herein.

FIG. 22A shows a first illustration, in perspective view, of an exemplary introducer for delivering a posterior TAM, per one or more embodiments herein.

FIG. 22B shows a second illustration, in top view, of an exemplary introducer for delivering a posterior TAM (e.g., the introducer of FIG. 22A), per one or more embodiments herein.

FIG. 22C shows a third illustration, inside view, of an exemplary introducer for delivering a posterior TAM (e.g., the introducer of FIG. 22A), per one or more embodiments herein.

FIG. 22D shows a fourth illustration, in top view, an exemplary introducer for delivering a posterior TAM (e.g., the introducer of FIG. 22A), per one or more embodiments herein.

FIG. 22E shows a fifth illustration, in top view, of an exemplary introducer for delivering a posterior TAM (e.g., the introducer of FIG. 22A), per one or more embodiments herein.

FIG. 23A shows a sixth illustration, in perspective view, of an exemplary introducer for delivering a posterior TAM (e.g., the introducer of FIG. 22A), per one or more embodiments herein.

FIG. 23B shows a seventh illustration, in perspective view, of an exemplary introducer for delivering a posterior TAM (e.g., the introducer of FIG. 22A), per one or more embodiments herein.

FIG. 23C shows an eighth illustration, in perspective view, of an exemplary introducer for delivering a posterior TAM (e.g., the introducer of FIG. 22A), per one or more embodiments herein.

FIG. 23D shows a ninth illustration, inside view, of an exemplary introducer for delivering a posterior Tracheal Access Mechanism (TAM) (e.g., the introducer of FIG. 22A), per one or more embodiments herein.

FIG. 24 shows a tenth illustration, in top view, of an exemplary introducer for delivering a posterior TAM (e.g., the introducer of FIG. 22A), per one or more embodiments herein.

FIG. 25A shows a first illustration, in perspective view, of an exemplary introducer for delivering an anterior TAM, per one or more embodiments herein.

FIG. 25B shows a second illustration, in perspective view, of an exemplary introducer for delivering an anterior TAM (e.g., the introducer of FIG. 25A), per one or more embodiments herein.

FIG. 25C shows a third illustration, in perspective view, of an exemplary introducer for delivering an anterior TAM (e.g., the introducer of FIG. 25A), per one or more embodiments herein.

FIG. 26A shows a first illustration, in perspective view, of an exemplary multibody introducer, per one or more embodiments herein.

FIG. 26B shows a second illustration, in perspective view, of an exemplary multibody introducer (e.g., the introducer of FIG. 26A), per one or more embodiments herein.

FIG. 27 shows additional illustrations, in perspective view, of an exemplary multibody introducer, per one or more embodiments herein.

FIG. 28A shows a first illustration, in perspective view, of an exemplary introducer with a flap, per one or more embodiments herein.

FIG. 28B shows a second illustration, in perspective view, of an exemplary introducer with a flap (e.g., the introducer of FIG. 28A), per one or more embodiments herein.

FIG. 28C shows a third illustration, in perspective view, of an exemplary introducer with a flap (e.g., the introducer of FIG. 28A), per one or more embodiments herein.

FIG. 29A shows a first illustration, in perspective view, of an exemplary introducer with a deployable flap, per one or more embodiments herein.

FIG. 29B shows a second illustration, in perspective view, of an exemplary introducer with a deployable flap (e.g., the introducer of FIG. 29A), per one or more embodiments herein.

FIG. 29C shows a third illustration, in perspective view, of an exemplary introducer with a deployable flap (e.g., the introducer of FIG. 29A), per one or more embodiments herein.

FIG. 30A shows a side view of an exemplary introducer with a low contact-angle flap, one or more embodiments herein.

FIG. 30B shows a side view of an exemplary introducer with a medium contact-angle flap, one or more embodiments herein.

FIG. 30C shows a side perspective view of an exemplary introducer with a high contact-angle flap, one or more embodiments herein.

FIG. 30D shows a side perspective view of an exemplary introducer with a higher contact-angle flap, one or more embodiments herein.

FIG. 31 shows a side perspective view of an exemplary Anterior Tracheal Access Mechanism (aTAM), per one or more embodiments herein.

FIG. 32 shows an illustration, inside view, perspective view, and front view, of an exemplary Anterior Shim (AShim), per one or more embodiments herein.

FIG. 33A shows a side perspective view of an exemplary aTAM trap door in a disengaged position, per one or more embodiments herein.

FIG. 33B shows a side perspective view of an exemplary aTAM trap door (e.g., the aTAM trap door of FIG. 33A) in an engaged position, per one or more embodiments herein.

FIG. 34A shows a perspective view of an exemplary aTAM trap door and an epiglottis in a disengaged position, per one or more embodiments herein.

FIG. 34B shows a perspective view of an exemplary aTAM trap door (e.g., the aTAM trap door of FIG. 34A) and an epiglottis in an engaged position, per one or more embodiments herein.

FIG. 35 shows a side view of an exemplary AShim having a noncolinear primary first segment and secondary first segment, per one or more embodiments herein.

FIG. 36A shows a side perspective view of an exemplary one-point bridge of an AShim, per one or more embodiments herein.

FIG. 36B shows a side perspective view of an exemplary two-point bridge of an AShim, per one or more embodiments herein.

FIG. 36C shows a top perspective view of an exemplary three-point bridge of an AShim, per one or more embodiments herein.

FIG. 37 shows a side perspective view of an exemplary AShim having a webbing, per one or more embodiments herein.

FIG. 38 shows side and side perspective views of an exemplary vine robot with a nub, per one or more embodiments herein.

FIG. 39 shows side views of an exemplary vine robot with a nub being redirected around the epiglottis, per one or more embodiments herein.

FIG. 40 shows side perspective views of an exemplary cable-actuated vine ETT, per one or more embodiments herein.

FIG. 41A shows a side perspective view of an exemplary passively controlled AShim in a deactivated configuration, per one or more embodiments herein.

FIG. 41B shows a side perspective view of an exemplary passively controlled AShim in an activated configuration, per one or more embodiments herein.

FIG. 42A shows a side perspective view of an exemplary actively controlled AShim in a deactivated configuration, per one or more embodiments herein.

FIG. 42B shows a side perspective view of an exemplary passively controlled AShim in an activated configuration, per one or more embodiments herein.

FIG. 43 shows side perspective views of an exemplary aTAM with a retractor, per one or more embodiments herein.

FIG. 44, shows side and side perspective views of an exemplary plunger disconnect be attached to a plunger, per one or more embodiments herein.

FIG. 45 shows side views of an exemplary automatic ETT deployment, per one or more embodiments herein.

FIG. 46 shows side and side perspective views of a laryngoscope with an exemplary vine robot, per one or more embodiments herein.

FIG. 47 shows section view diagrams of an exemplary method for intubating a patient, per one or more embodiments herein.

FIG. 48 shows side view illustrations of an exemplary toroidal vine robot transmitting an ETT, per one or more embodiments herein.

FIGS. 49A-49C show perspective views of a tracheal intubation device including a introducer and a tracheal access mechanism (TAM) including a base having an aperture for passing an endotracheal tube (ETT) subassembly, a bridge, and a flexible datum according to an exemplary embodiment.

FIG. 50 shows a rear view of a TAM including an ETT egress port offset and a datum having upper esophageal sphincter (UES) self-centering width and molar avoidance geometry according to an exemplary embodiment.

FIGS. 51A-51B show perspective (FIG. 51A) and side (FIG. 51B) views of an introducer including a connected member, two unconnected members, and a TAM according to an exemplary embodiment.

FIGS. 52A-52B show front (FIG. 52A) and rear (FIG. 52B) perspective views of an introducer including a directionally-compliant portion along the length of the introducer configured to bend easily when in contact on a posterior surface and to be rigid in contact on an anterior surface in accordance with example embodiments described herein.

FIGS. 53A-53B show schematic views of an introducer including a directionally-compliant portion including series of grooves configured to enhance bending in a preferential direction in accordance with example embodiments described herein.

FIGS. 53C-53D show schematic views of an introducer including a directionally-compliant portion including a series of pillars configured to enhance bending in a preferential direction in accordance with example embodiments described herein.

FIGS. 54A-54B show perspective views of a tracheal intubation device including a removable display or video monitor, and a connector configured to releasably interface the video monitor with an introducer including a TAM and an interface having an ETT egress according to an exemplary embodiment.

FIGS. 54C-54D show perspective views of the tracheal intubation device of FIGS. 54A-54B including a removable ETT subassembly according to an exemplary embodiment.

FIGS. 55A-55F show top-down (FIGS. 55A-55B), side (FIGS. 55C-55D), and perspective (FIGS. 55E-55F) views of a tracheal intubation device including an introducer having active actuation configured to change a radius of curvature along a body of the introducer to change with applied forces according to an exemplary embodiment.

FIG. 56A shows a perspective view of a introducer including a pair of unconnected members connected to a TAM and loaded with an ETT according to an exemplary embodiment.

FIGS. 56B-56C show rear (FIG. 56B) and front perspective (FIG. 56C) views of a connected introducer including a pair of lateral rails configured to contact posterior tissue and preserve a volume between connected members, and a connected anterior surface configured to support a datum according to an exemplary embodiment.

FIG. 57 shows a perspective view of a connected introducer including a pair of unconnected members each having a centering pin on a distal end according to an exemplary embodiment.

FIGS. 58A-58C show perspective (FIG. 58A) and side (FIGS. 58B-58C) views of an introducer including a posterior valence mechanism or floating connectors configured to induce passive actuation by augmenting curvature of a body of the introducer and induce tension on a posterior side of the TAM in accordance with example embodiments described herein.

FIGS. 59A-59D show alternate views of an introducer according to an exemplary embodiment.

FIGS. 60A-60B show alternate views of an introducer including a gastric port according to an exemplary embodiment.

FIGS. 61A-61B show alternate views of a tracheal intubation device including an introducer with an assembled ETT subassembly coupled to the introducer according to an exemplary embodiment.

FIG. 62 shows a tracheal intubation device including an introducer having proximal and distal retention features with an ETT subassembly having complementary proximal and distal retention features according to an exemplary embodiment.

FIG. 63A shows distal retention features of the introducer according to an exemplary embodiment.

FIG. 63B shows a ETT subassembly secured within the distal retention features of the introducer according to an exemplary embodiment.

FIG. 63C shows a ETT subassembly including a proximal retention feature aligned with a proximal retention feature of the introducer according to an exemplary embodiment.

### DETAILED DESCRIPTION

Provided herein are novel airway management devices, systems, and methods that facilitate and autonomously perform endotracheal intubation using a vine robot. In some embodiments, the devices herein exploit knowledge of a patient's anatomy to establish tissue conditions which enable vine robot endotracheal intubation.

An introducer subassembly (“introducer”) can be a user-interfacing enclosure designed to facilitate the correct placement of the total device and vine robot. The tracheal access mechanism (“TAM”) can locate the airway, establish requisite tissue conditions, and place the vine robot in the proper initial position. In some embodiments, a vine ETT subassembly (“vine robot”) is configured to navigate the airway without a visualization system via self-expanding soft geometry and provides a sealed and direct airway for ventilation.

In some cases, a method of using these devices rely on tissue interaction to properly advance the TAM to the hypopharynx and can include a continuous connection between the TAM and the advancement feature (in these cases a handle for manual advancement).

In some embodiments, the systems herein comprise an introducer comprising a user-interfacing enclosure designed to facilitate the correct placement of the device and vine robot during intubation. The devices, systems, and methods herein can be configured to perform airway management, diagnostic and therapeutic endoscopy (across the human body), and general autonomous endoluminal access. In some embodiments, the devices and methods herein employ introducers, sliding shims (advanced both automatically and manually), self-locating features, and Endotracheal Tube (ETT) advancement mechanisms for tracheal intubation. In some embodiments, the vine robot navigates a patient's airway without the need for a visualization system, wherein its self-expanding soft geometry provides a sealed and direct airway for ventilation. In some embodiments, the devices and systems herein can be employed with steering, image guidance (e.g. guided in real time by imaging studies including CT, ultrasound, or MRI), or designed to follow a predetermined path through the body to the target (e.g. designed using pre-procedure imaging studies to follow a path to the target). In some embodiments, the vine robot herein is composed of metals, plastics, or both. The devices and systems herein may employ multiple concentric pathways or lumens for instrumentation.

In some embodiments, the everting growth technology of the vine robots herein are employed for surgical resection of tumors throughout the body, including in the lung periphery, pleural space, or in the trachea or bronchi, including partial lobectomies and wedge or segmental lung resection, and in the gastrointestinal or reproductive tracts. The vine robots herein can be used in mediastinoscopy and/or be coupled with video-assisted thoracoscopic surgery (VATS) technology for partial lung resection, lung biopsies, pleural biopsies, or any combination thereof. The vine robots herein can be coupled with existing robotic technology (e.g., DaVinci platforms) for robotic surgery throughout the body (e.g., the thorax, pleural space, lung, abdomen, head, neck, and bladder). The vine robots herein can be employed with any current endoscopy procedure such as colonoscopies, laryngoscopies, and bronchoscopies. The vine robots herein can be used to transport a camera or biopsy/surgical instruments within a patient.

Vine robots suitable for use with the various embodiments described herein, as well as accessory devices suitable for such vine robots and their uses, are further described in U.S. patent application Ser. No. 17/632,335, filed Feb. 2, 2022 and entitled “Vine robot tracheal intubation device” and PCT Application No. PCT/US2022/044162, filed Sep. 20, 2022 and entitled “Soft Robot Intubation Device”, which are incorporated herein by reference.

### Airway Management

Disclosed in this document are systems, devices, and methods to pin an epiglottis of a patient (e.g., epiglottis defeat) during soft robotic intubation. Pinning the epiglottis of a patient anteriorly to facilitate the passage of an intubating vine robot can be achieved by the devices and systems herein though manual epiglottis defeat, wherein a tool is progressed using human action, or autonomous epiglottis defeat configuration, wherein a tool is progressed through soft robotic actuation).

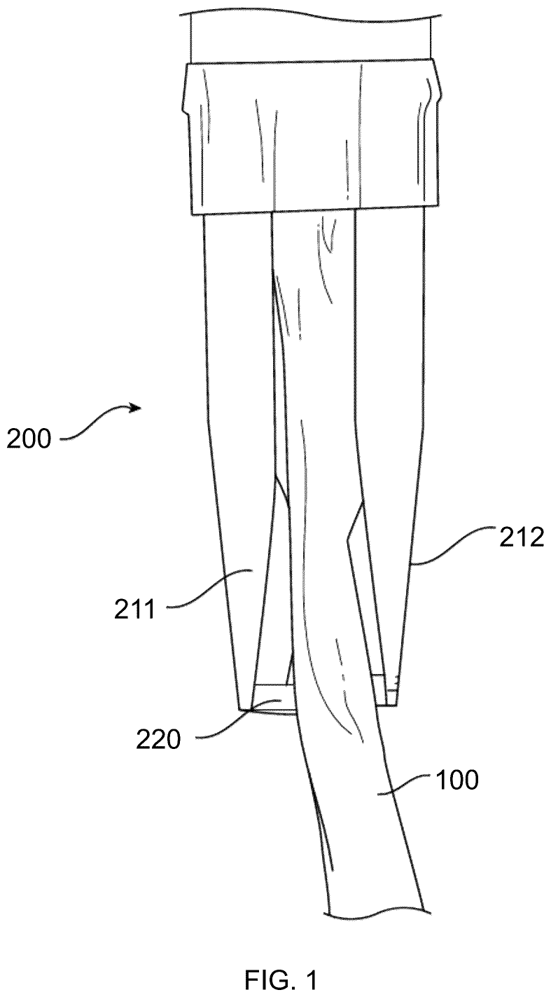

FIGS. 1-3 show exemplary shims 200 for insertion of a vine robot 100. As shown, the shim 200 may comprise a first tine 211, a second tine 212, and a rail 220. In some embodiments, passive pinning stabilizes the epiglottis without the requirement for subsequent action. In one example, the shim 200 effectively pins the epiglottis during deployment. Per FIG. 1, the rail 220 may be parallel to the shim tines 211 212 and can be slightly narrower than an outer diameter of the vine robot 100. In some embodiments, the rail 220 aids in pinning the epiglottis, wherein once the shim 200 defeats the epiglottis, the rails 220 maintain contact with the tongue and the epiglottis, allowing the vine robot 100 to ride along a posterior surface of the shim 200. In some embodiments, with a slight anterior valence, this vine robot 100 can pass through the tines 211 212 to intubate at the appropriate depth. In some embodiments, the first tine 211 and the second tine 212 enable individual contact of the shim 200 with a patient's variable posterior pharyngeal wall anatomy. In some embodiments, a separation between the first tine 211 and the second tine 212 forms a cavity through which the vine robot 100 can pass to access the trachea.

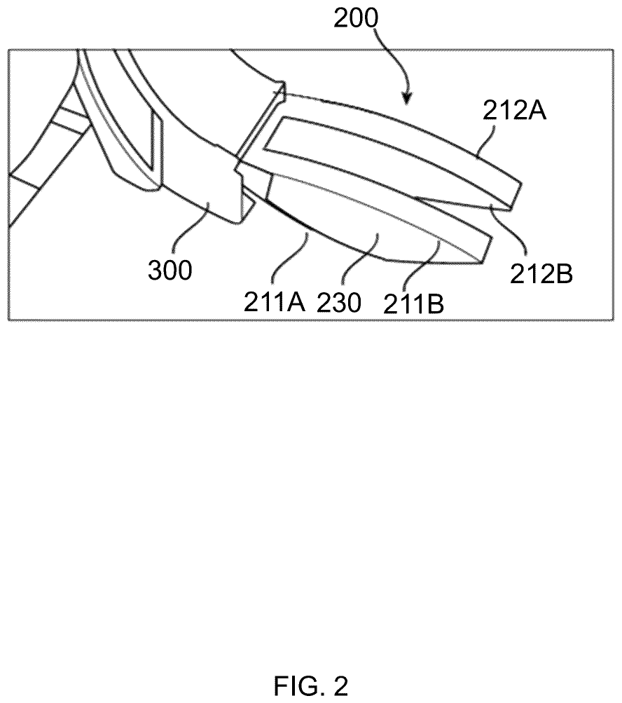

In some embodiments, per FIG. 2, the first tine 211 comprises a first anterior tine layer 211A and a first posterior tine layer 211B and the second tine 212 comprises a second anterior tine layer 212A and a second posterior tine layer 212B. In some embodiments, driving the shim 200 posteriorly to the epiglottis displaces the anterior shims 211A 212A from the posterior shims 211B 212B. In some embodiments, the anterior shims 211A 212A and the posterior shims 211B 212B are flexible. In some embodiments, the anterior shims 211A 212A and the posterior shims 211B 212B are separated by a spring layer 230. In some embodiments, the spring layer 230 comprises foam, a spring, a flexure, or any combination thereof. In some embodiments, ejecting the shim 200 from an introducer 300 allows the spring layer 230 to separate the anterior shims 211A 212A and the posterior shims 211B 212B, wherein the first anterior tine layer 211A and the second anterior tine layer 212A hold the tongue and epiglottis in place.

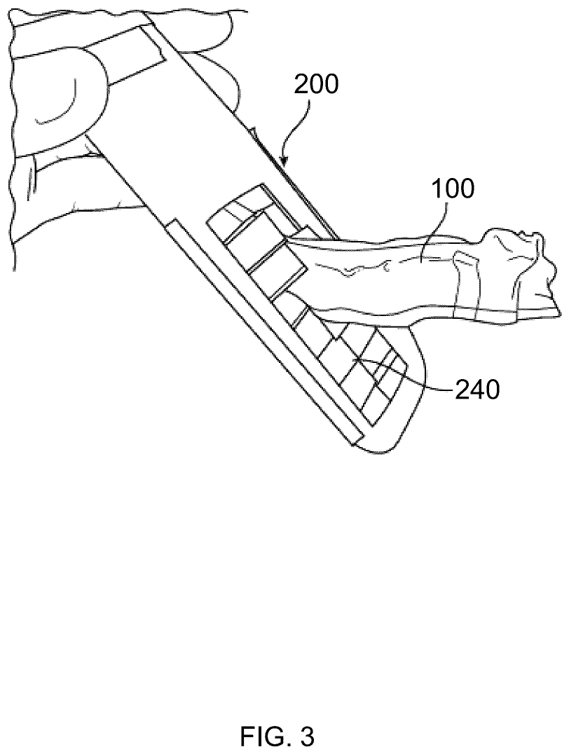

As shown in FIG. 3, the shim 200 may comprise a plurality of overlapping flaps 240. In some embodiments, when the shim 200 is inserted into the mouth of a patient, the flaps 240 are configured contact the back of the tongue and epiglottis after epiglottis defeat. In some embodiments, the flaps 240 have a geometry and are formed of a material configured to minimize traction against the patient's tissue. Once in position, the vine robot 100 may be deployed between a portion of the flaps 240 not held in place by the weight of the tissue to enter the trachea.

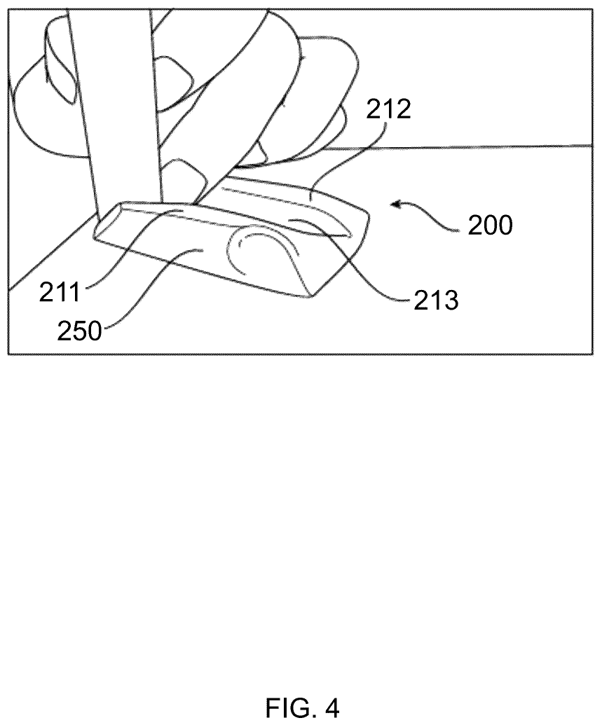

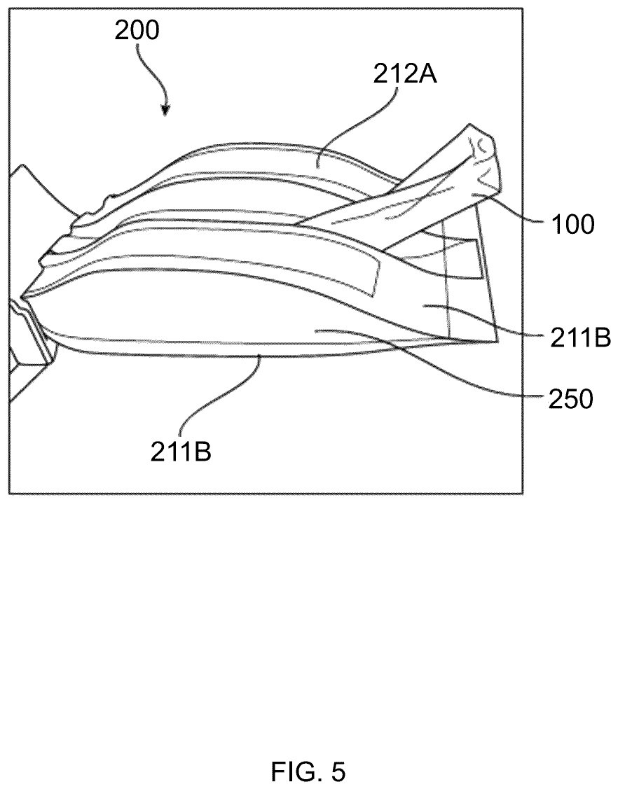

In some embodiments, per FIGS. 4-5, the shim 200 can be configured for active pinning of the epiglottis after epiglottis defeat. As shown, the shim 200 can comprise an inflatable pinning body 250, which lift the shim tines 211A 211B 212A 212B to actively pin the epiglottis. In some embodiments, once the inflatable pinning body 250 is filled, a channel 213 is formed between the tines 211 212 through which the vine robot may pass. In some embodiments, inflatable pinning body 250 has a greater height than width. In some embodiments, the inflatable pinning body 250 translates the first anterior tine layer 211A, the first posterior tine layer 211B, the second anterior tine layer 212A, the second posterior tine layer 212B, or any combination thereof.

### Endoscopy

Endoscopy in humans can be performed by guiding, by a medical practitioner, a rigid scope with both a camera and a working channel through a lumen of the human body. The vine robots herein can enable and improve diagnostic and therapeutic methods in endoscopic applications such as, for example, laryngoscopy, bronchoscopy, gastroscopy, enteroscopy, colonoscopy, cystoscopy, gynecological endoscopy, and vascular endoscopy.

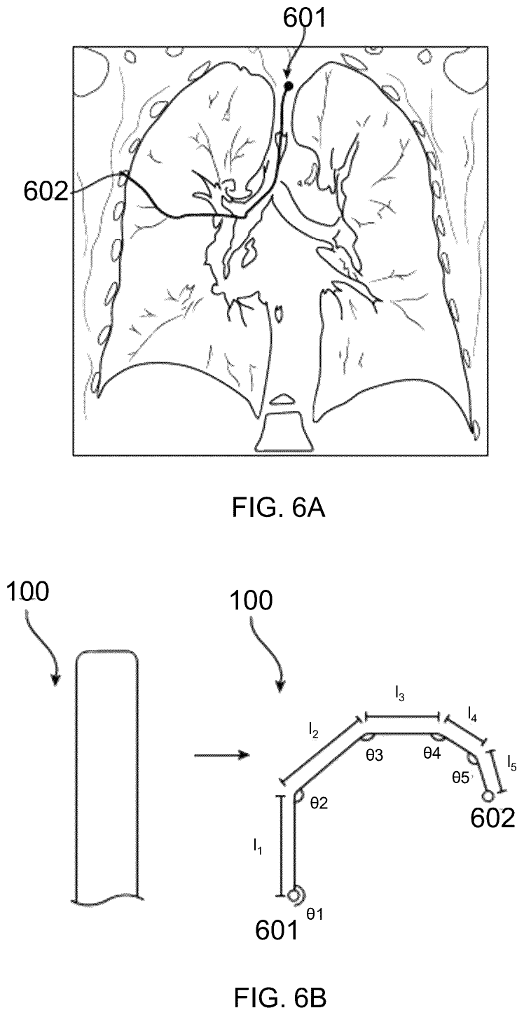

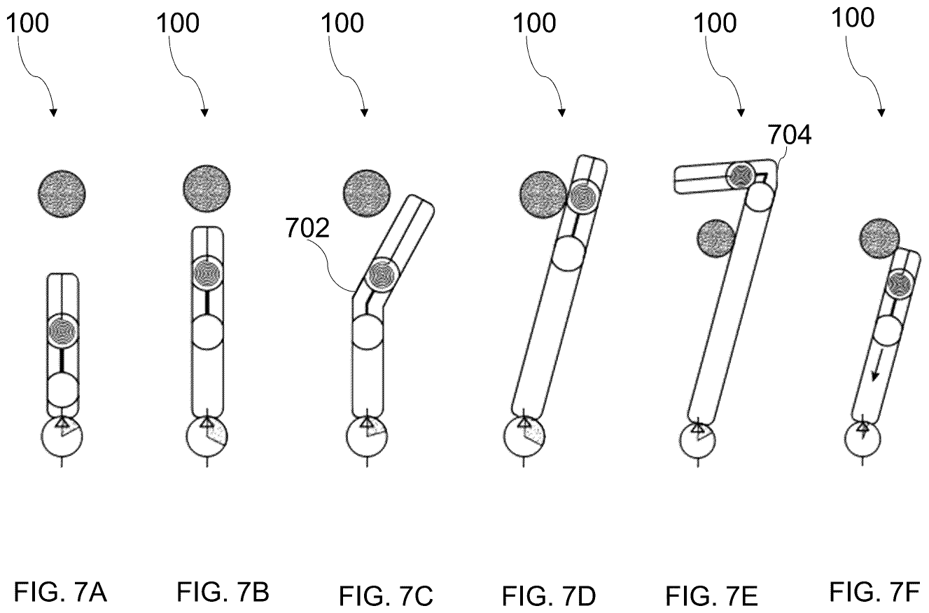

In some embodiments, the vine robots 100 herein progress by add material to its proximal patient-side tip, which enables a vine robot 100 with built-in navigation features to alter its path as it grows. FIG. 6A shows an exemplary map of a human airway. In some embodiments, a lumen of the human body comprises, for example, an airway, a vascular system, biliary system, a hepatobiliary tract. In some embodiments, the vine robots herein are shaped to follow a specific path having a starting point 601 and an end point 602 through the human body lumen per relevant measurements of the lumen. In some embodiments, per FIG. 6B, when dimensions of the lumen are known, segment lengths 11, 12, 13, 14, 15 and angles θ1, θ2, θ3, θ4, θ5 can be discerned by, for example, 3D imaging. The vine robot 100 may include features according to these lengths and angles, which when inverted, can be deployed through the lumen. In some embodiments, the devices herein further comprise a working channel, a camera, or both. In some embodiments, the vine robot 100, when inflated, is configured to have a plurality of segment lengths and angles between segments that match lengths and curvatures of a predefined bodily lumen. In some embodiments, per FIGS. 7A-7F show exemplary steps in a point-controlled method of navigating a vine robot through a lumen and around an obstacle. FIGS. 7A-7B show that the extension of the vine robot 100 can directly approach the obstacle. FIG. 7C shows the vine robot 100 can bend at a junction 702 of the vine robot at a desired angle θ1. FIG. 7D shows the vine robot's ability to adjust the first portion of the vine robot 100 to match the angle θ1 of the second portion. The vine robot 100 can be advanced tangentially past the obstacle. The vine robot can bend at a junction 704 at a desired angle θ2 as shown in FIG. 7E. The junction 704 can be located in a similar, same, or different location than the junction 702. θ1 can be the same, similar, or different angle from θ2 depending on the medical practitioner's strategy for advancing past the obstacle. As shown in FIGS. 7C and 7E, θ1 and θ2 can be different, including in different directions. Once the obstacle has been navigated past and the procedure is complete, the vine robot 100 can be withdrawn at the same or different path than it was intubated, as shown in FIG. 7F.

In some embodiments, provided herein is a soft vine endoscopy robot 100, comprising a primary tube body 110 having a sealed first end 111 and a second end 112 opposite the first end 111. In some embodiments, the first end 111 is within a mesial portion 130 of the tube body 110.

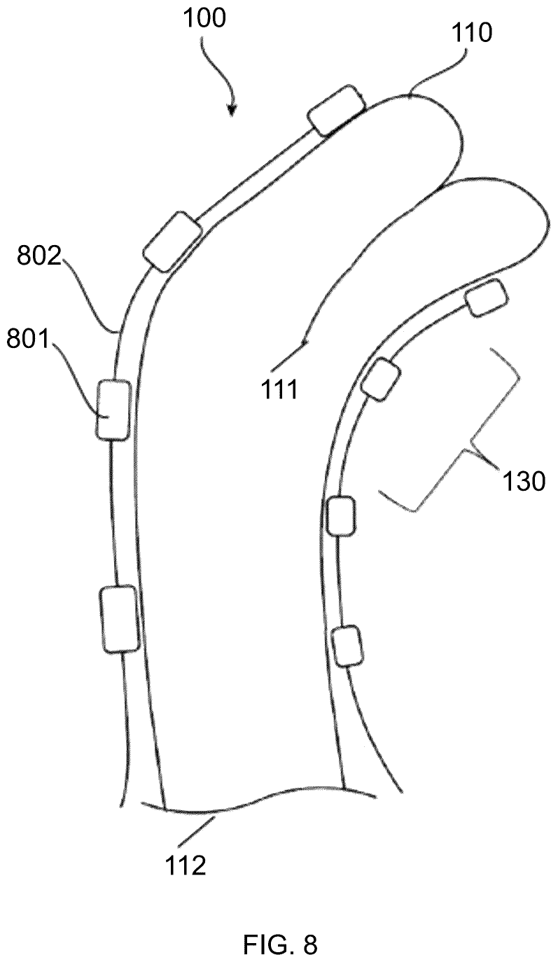

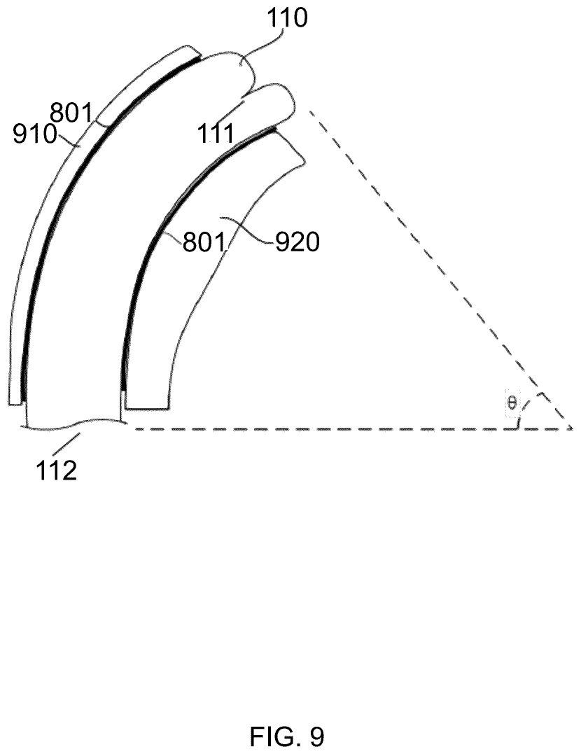

In some embodiments, per FIGS. 8-9, the tube body 110 comprises a plurality of segments separated from one another by the two or more stiffened portions 801. In some embodiments, the primary tube body 110 comprises two or more stiffened portions 801. In some embodiments, adjacent stiffened portions 801 are separated by a segment of the primary tube body 110. In some embodiments, the two or more stiffened portions are arranged from the first end to the second end. In some embodiments, the two or more stiffened portions 801 are radially aligned about the tube body 110. In some embodiments, the two or more stiffened portions 801 are radially arrayed about the tube body 110. In some embodiments, the two or more stiffened portions 801 have a thickness greater than the rest of the tube body 110. In some embodiments, the two or more stiffened portions 801 have a modulus of elasticity less than the rest of the tube body 110.

In some embodiments, per FIG. 8, the two or more stiffened portions comprises a stop coupled to the tube body. In some embodiments, the stiffened portions 801 comprise a stop wherein two or more stops are interconnected by a tendon 802. In some embodiments, the tendon 802 has a constant length. In some embodiments, the tendon 802 has an actuatable length. In some embodiments, actuation of the tendon 802 and/or pneumatic actuation of the primary tube body 110 can be used to control the relative lengths of sides the vine robot 100 to form a constant curve or buckle the body to produce a point curvature. In some embodiments, pneumatic actuation employs pneumatic artificial muscles, inverse pneumatic artificial muscles, or both (e.g., a Mckibben actuator).

In some embodiments, when inflated, the tube body 110 has two non-colinear sections 910 920 that intersect at one of the two or more stiffened portions 801. In some embodiments, per FIG. 9, the tube body 110 comprises a first non-colinear section 910 that intersects the tube body 110 at a first stiffened portion 801 and a second non-colinear section 920 that intersects the tube body 110 at a second stiffened portion 801. In some embodiments, inflating the first non-linear section 910 bends the tube body 110 at an angle (θ) towards the first non-linear section 910. In some embodiments, inflating the second non-linear section 920 bends the tube body 110 at an angle towards the second non-linear section 920.

### Visualization

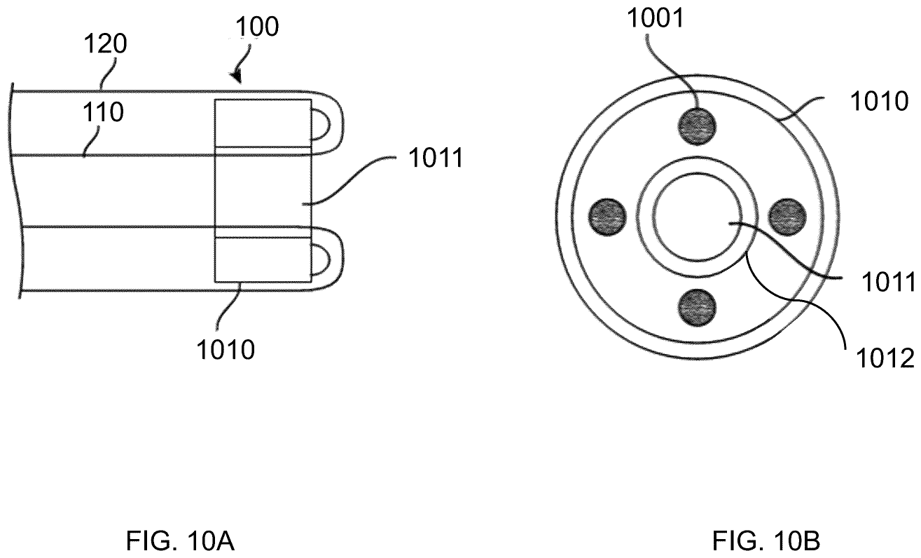

As the vine robot herein can employ tip-based material addition, per FIGS. 10-11, a sensor 1001 may be coupled to the tip, within its body, or both for visualization.

In some embodiments, per FIGS. 10A-10B, the vine robot 100 further comprises an annular chassis 1010 having a cavity 1011. In some embodiments, per FIG. 10B, the vine robot 100 extends from its first end 110, through the cavity 1011 with outer wall 1012, around an outer surface of the annular chassis 1010 and to the second end 120. In some embodiments, the sensor 1001 comprises a camera sensor an ultrasound sensor, or both.

In some embodiments, per FIG. 11, the vine robot 1000 further comprises a camera body 1020 comprising the sensor 1001. In some embodiments, camera body 1020 comprises a distal portion 1021 comprising one or more sensors 1001, a proximal portion 1023 having an outer diameter greater than an inner diameter of the annular chassis 1010, and a mesial portion 1022 (not shown) between the distal portion 1021 and the proximal portion 1023. In some embodiments, the mesial portion 1022 is sized to fit within the cavity 1011 of the annular chassis 1010.

In some embodiments, the vine robot 100 herein employs two or more sensors 1001 for visualization. In some embodiments, the two or more sensors 1001 are arrayed along the vine robot 100, which when evert, capture location-based sensor data to map in a discrete fashion. In some embodiments, the sensors 1001 are connected to a base station in a wireless or wired fashion.

In some embodiments, the tip-based material addition of the vine robot 100 covers the sensor 1001 with a clean film barrier during translation of the vine robot 100. In some embodiments, tip-directed friction force produced through this tail passage through the center of the annular chassis 1010 self-rightens of the annular chassis 1010 and the sensor 1001.

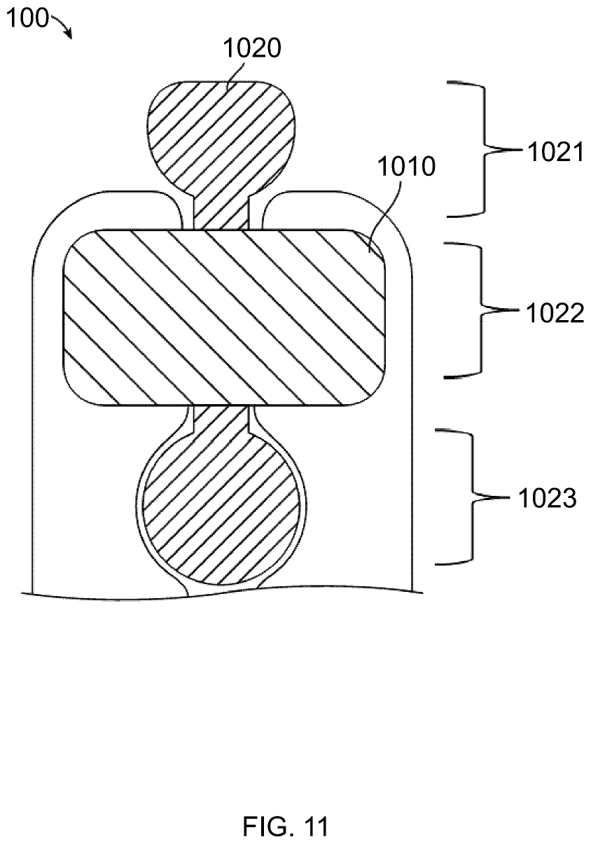

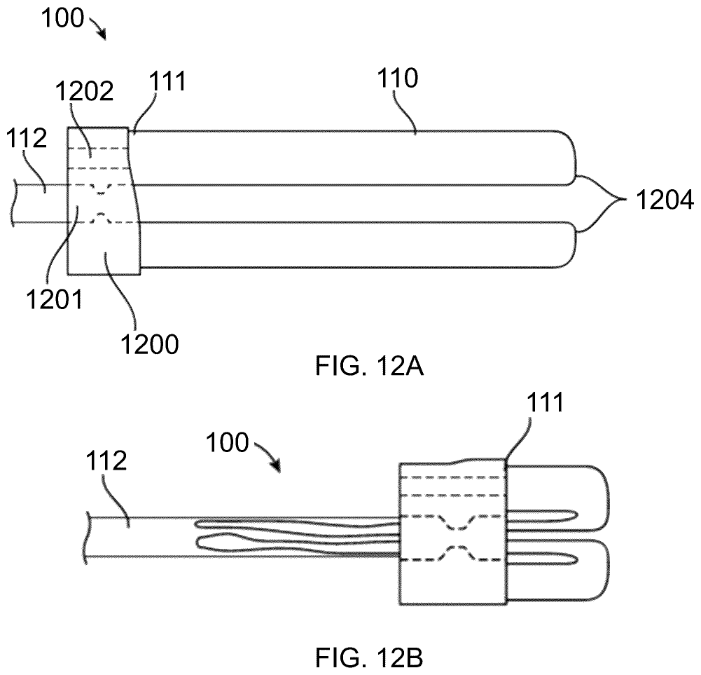

Such a sensor can be used to increase the efficacy of any endoscopic tool. As such, in some embodiments, per FIGS. 12A-12B, the vine robot 100 comprises an endoscope connector 1200. In some embodiments, the endoscope connector 1200 comprises an endoscope fastener configured to fasten to an endoscope. In some embodiments, the endoscope connector 1200 comprises a converging-diverging hole 1201, and an inflation port 1202. In some embodiments, a first end 111 of the primary tube body 110 is coupled and sealed to an outer surface of the endoscope connector 1200. In some embodiments, a sealed second end 112 of the primary tube body 110 passes through the converging-diverging hole 1201. In some embodiments, inflating the primary tube body 110 through the inflation port 1202, from an uninflated state per FIG. 12B to an inflated state per FIG. 12A, extends a distal surface of the primary tube body 110 away from the endoscope connector 1200. In some embodiments, maintaining a set pressure within the primary tube body 110 seals a mesial portion of the primary tube body 110 against the converging-diverging hole 1202.

In some embodiments, the vine robots herein can integrate with existing robotic technologies to facilitate multi-modal functionality. Additionally, the vine robots herein can couple to existing cameras, robotic devices, or biopsy technology to deliver an intervention to, for example, an endobronchial tumor or target via a simple connector and everting tool channel assembly. In some embodiments, the vine robot is coupled to an ion robotic-assisted bronchoscopy platform (e.g., Intuitive) for lung biopsies. In another embodiment, the vine robot is coupled to a Monarch robotic-assisted bronchoscopy platform (e.g., Auris)

### Retraction

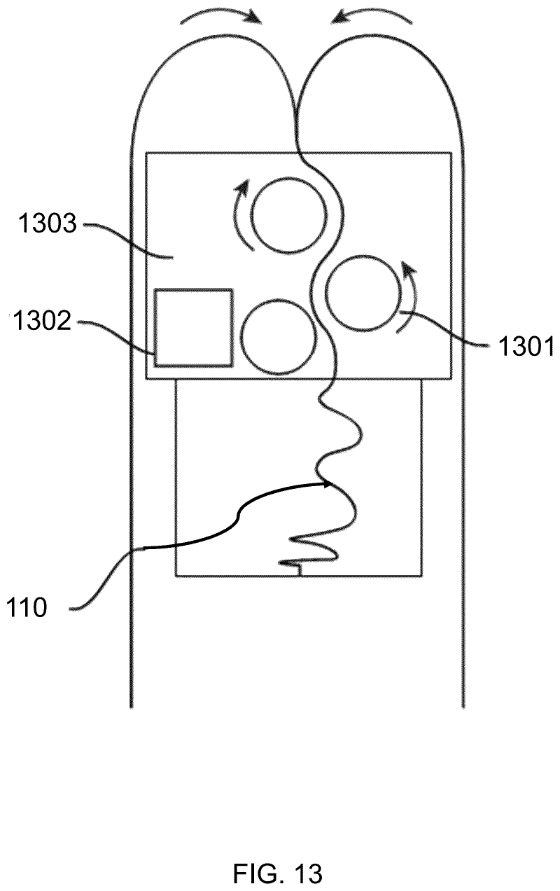

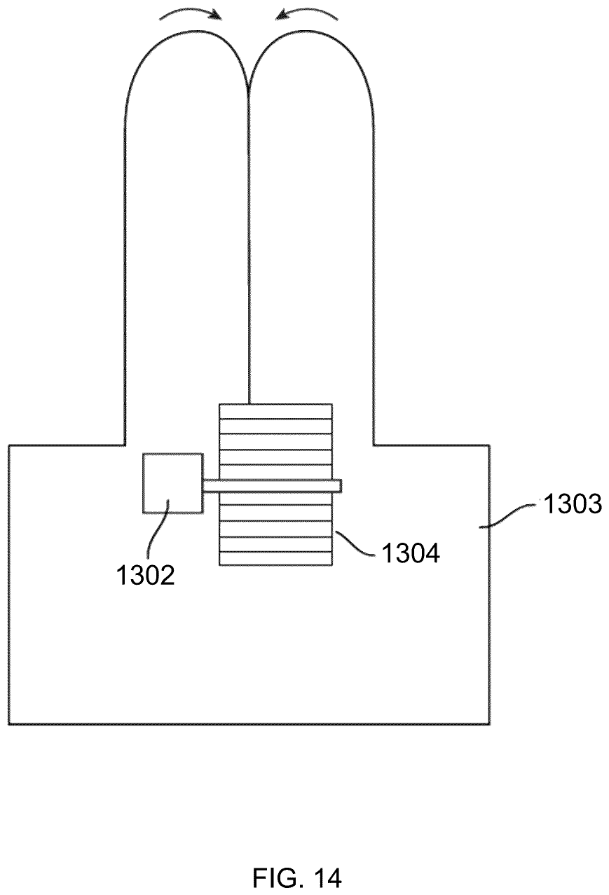

In some embodiments, the tube body of the vine robot can be retracted for removal from a patient. In some embodiments, per FIGS. 13-14, retraction is tip-based. In some embodiments, per FIG. 13, one or more rollers 1301 driven by a motor 1302 collect the tube body 110. In some embodiments, the one or more rollers 1301 collect the tube body 110 into a housing 1303. In some embodiments, the one or more rollers 1301, the motor 1302, or both are contained within the housing 1302. In some embodiments, per FIG. 14, the tube body is collected on a reel 1304. In some embodiments, the reel 1304 is driven by a motor 1302. In some embodiments, the reel 1304, the motor 1302, or both are within a housing 1303.

### Tool Channels

In some embodiments, multi-walled everting vine robots with multiple channels therebetween easily couple with an endoscope. If one channel is desired, a double-walled device can be incorporated. If multiple channels are desired, the second wall can be internally segmented to facilitate more channels, or additional double-walled devices of smaller diameter can be deployed within the original device (as a nested deployment).

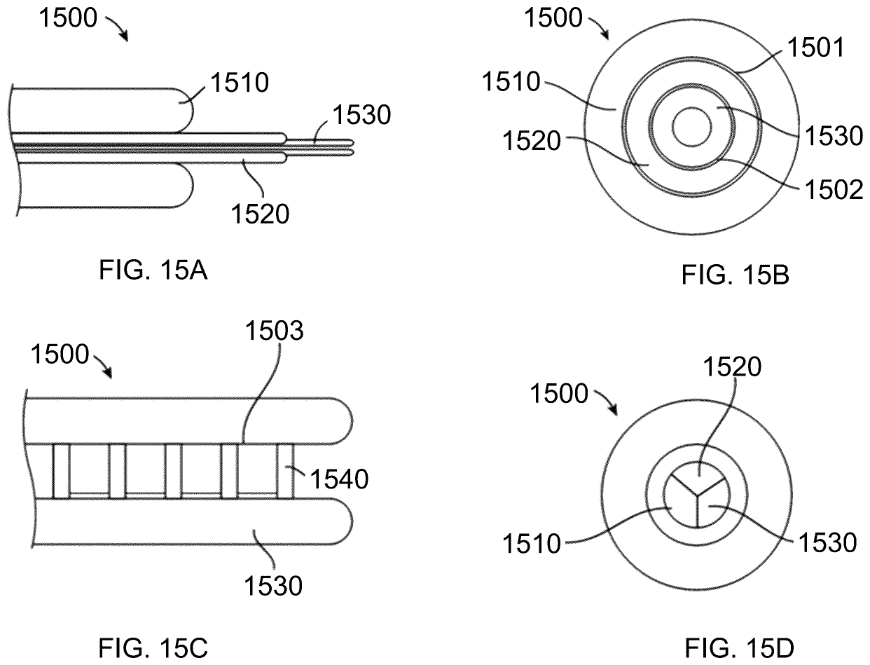

FIGS. 15A-15D show exemplary multi-walled vine robots 1500. In some embodiments, per FIGS. 15A-15B, the multi-walled vine robot 1500 comprises an outer tube body 1510, a middle tube body 1520, and an inner tube body 1530, wherein the inner tube body 1530 is within a middle channel 1502 by the middle tube body 1520, and wherein the middle tube body 1520 is within an outer channel 1501 of the outer tube body 1510. In some embodiments, per FIG. 15C, an inner channel 1503 of the inner tube body 1530 comprises one or more reinforcements 1540. In some embodiments, per FIGS. 15A-15C, the outer channel 1501, the middle channel 1502, and the inner channel 1503 are concentric. In some embodiments, per FIG. 15D, the outer channel 1501, the middle channel 1502, and the inner channel 1503 are radially arrayed.

### Introducer

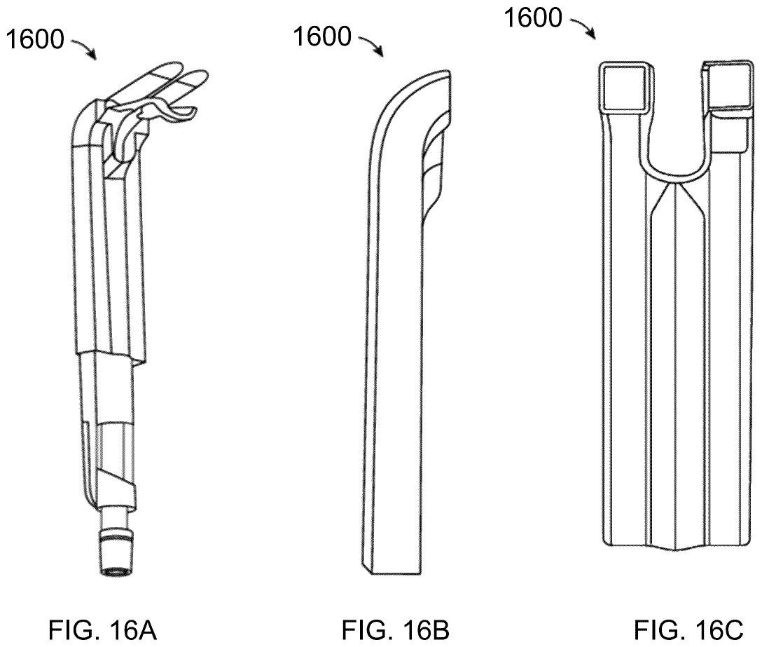

Introducers provided herein can enable blind intubation on patients with wide variations in anatomy. In some embodiments, the introducers are configured to be naturally introduced to an anatomical position, such as a laryngeal mask airway (LMA), to reduce a length between the anatomical position and an intubation position. In some embodiments, per FIGS. 16A-16C, an orotracheal intubation introducer 1600 reliably locates the oropharyngeal wall. Alternatively, introducers can be used for nasotracheal intubation to aid in the location of, for example, the inferior turbinates. FIG. 16A shows a perspective view, FIG. 16B shows a side view, and FIG. 16C shows a front view of introducer 1600.

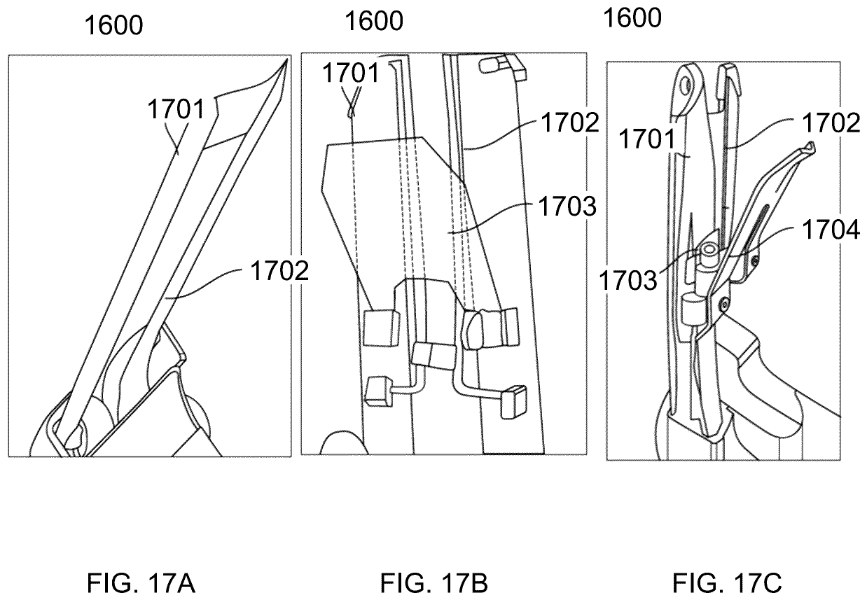

Per FIGS. 17A-17C, the introducers 1600 herein may comprise a shim 1701 1702 to prevent a blind device from strafing the pharyngeal wall as it advances through the oropharynx and into the upper airway. The introducer 1600 may comprise a first shim 1701 and a second shim 1702 to prevent contact of the introducer with the C spine to enable deflection into the upper airway. In some embodiments, the introducer 1600 is advanced manually. In some embodiments, the introducer 1600 is advanced automatically using, for example, a motor an electronic actuator, a magnetic field generator, pneumatics, or hydraulics. The shims 1701 1702 may be integral to the introducer 1600 (e.g., for an LMA-type introducer design), or disparate from the introducer 1600. The shims 1701 1702 may comprise a passive lifting mechanism (e.g., a foam or spring), an active lifting mechanism (i.e., pneumatics), or both, to lift the anatomy once in place. In some embodiments, the introducer 1600 comprise a ramp or staircase to displace the anatomy to different levels through advancement as desired. In some embodiments, per FIGS. 17B-17C, respectively, the introducer 1600 herein comprises flap 1703 and a rail 1704, to pin the epiglottis in place. In some embodiments, per FIG. 17A, the introducer comprises a camera or fiber optic bundle 1703 to visualize the placement of the shims 1701 1702 and tissue state during operation.

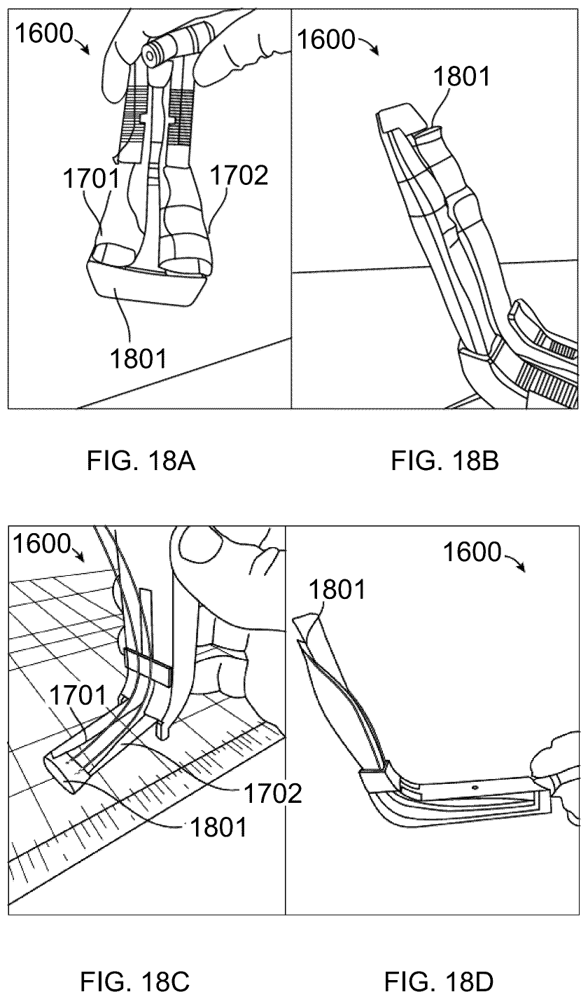

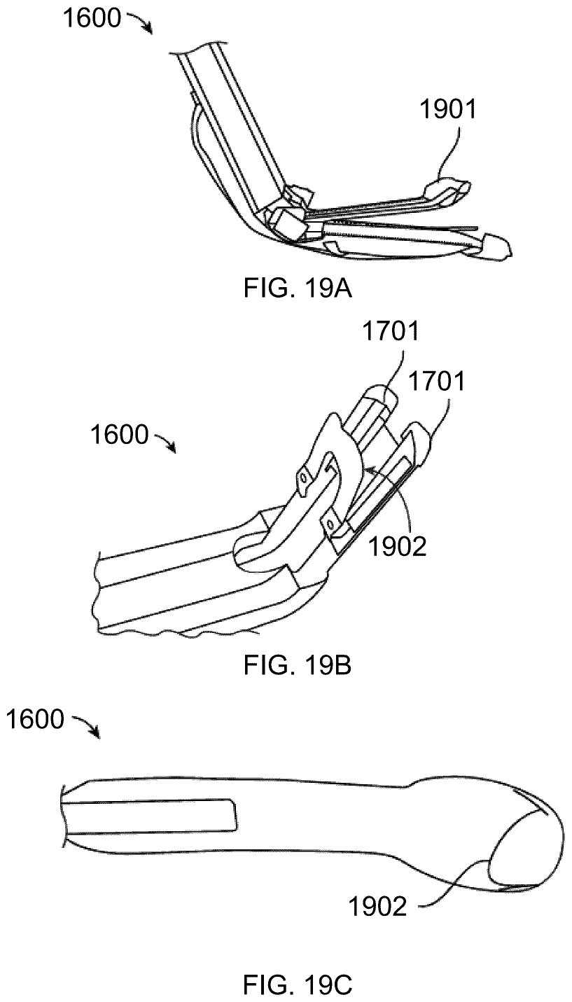

In some embodiments, the shims 1701 1702 of the introducer 1600 are configured to stop insertion once an operative anatomical position is reached. Per FIGS. 18A-18D, the introducer may comprise a wedge 1801 at a leading edge of the shim assembly to locate the glottis and arytenoids. FIGS. 19A-19C show a shim with a vallecula stop 1901 1902 to prevent the shim from advancing past the vallecula and/or glottic opening. In some embodiments, per FIG. 19A, the vallecula stop 1901 is separated from the shim, wherein per FIGS. 19B-19C, the vallecula stop 1902 is integrated into the shims 1701 1702. In some embodiments, the vallecula stop 1902 comprises a force-limiting advancement mechanism (e.g., a spring or a flexure) to minimize trauma to the anatomy. Such introducers 1600 may alternatively be configured for application to other areas of the body (e.g., the urethra, the bladder, the main stem bronchi, or the bronchioles).

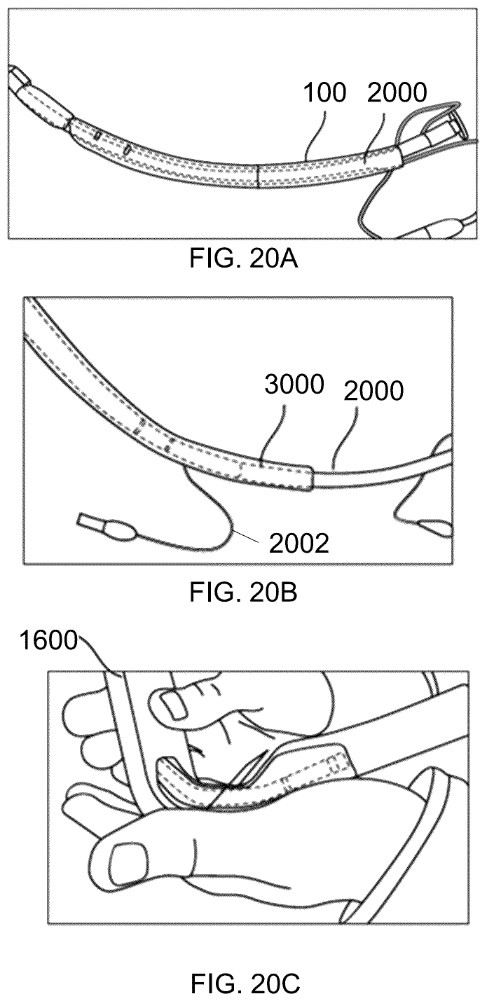

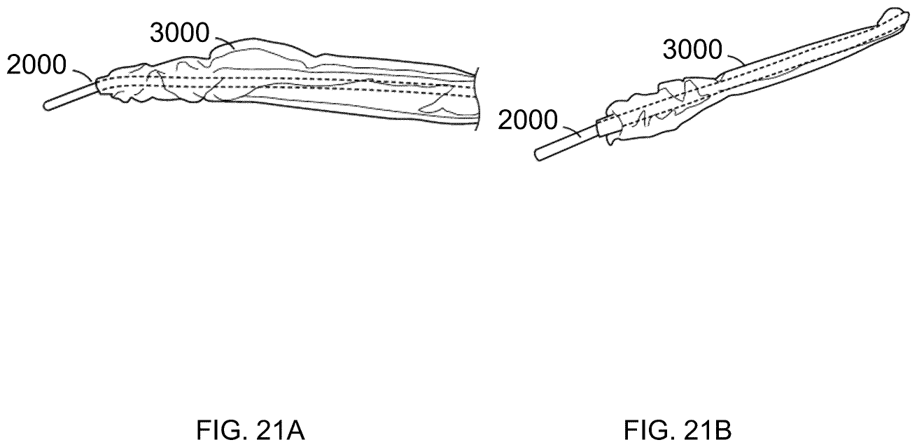

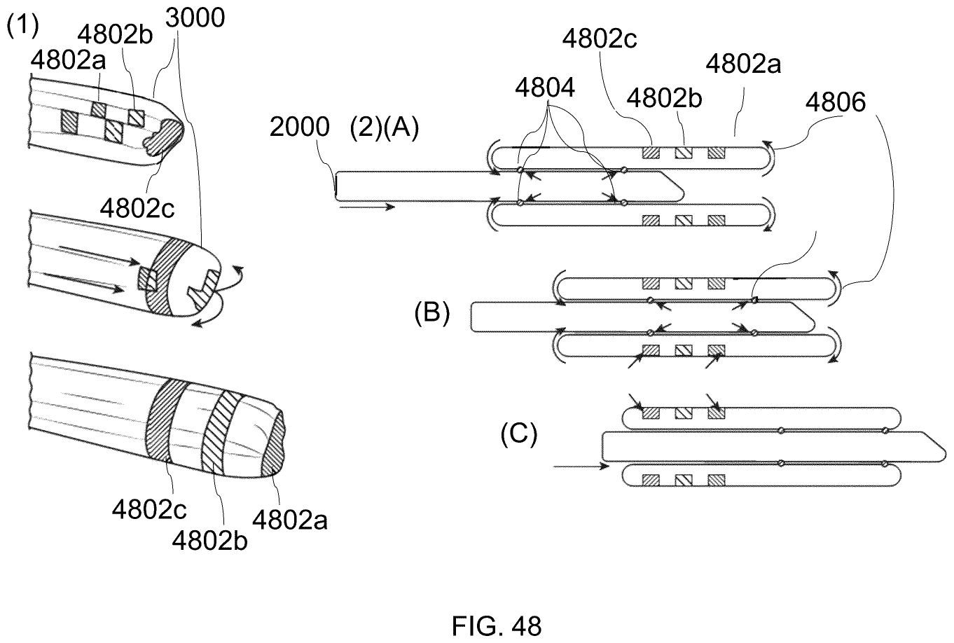

In some embodiments, once the introducer is advanced and the vine robot is placed, an Endotracheal Tube (ETT) is passed through the vine robot for successful tracheal intubation. In some embodiments, per FIGS. 20A-20C, the ETT 2000 is sheathed within the vine robot 100. In some embodiments, per FIGS. 20B and 48, the vine robot is a toroidal vine robot 3000, wherein a constant volume of fluid is maintained therein. In some embodiments, the vine robot may include an inflation line 2002. In some embodiments, the fluid comprises air, water, oil, or any combination thereof. In some embodiments, the toroidal vine robot 3000 is coupled to the ETT 2000. In some embodiments, the toroidal vine robot 3000 is disparate from the ETT 2000. In some embodiments, pull-tendons may be incorporated to guide this assembly under direct visualization or routed through to the base of the introducer for provider control. FIGS. 21A-21B show a commercial ETT sheathed in a toroidal vine robot 3000.

It may be more economical to sheath an ETT in a vine robot to advance it via growth. This may be best advanced in a toroidal sheath. The toroidal sheath may be a constant length and concurrently evert and invert material at either end, or use a rolling diaphragm that serves to lengthen the device continuously. Both may use some form of insufflation; in the toroidal case, a constant volume of air is used to maintain a constant pressure (as the overall volume does not change), while in the lengthening case additional air is required to maintain a constant pressure. A camera may be affixed to this assembly, either internally or externally, to guide it.

FIG. 48 shows the emergence and eversion of toroidal vine robot 3000. Three areas of the outer tubing can be represented by the three types of shading 4802a, 4802b, and 4802c. These can be different materials or ways to show different sections of the same material of the outer tubing. The left progression (1) shows an external view of the material emerging and everting 4806, supported by the reversal of the order of 4802a, 4802b, and 4802c. The right progression (2) shows a schematic internal view of the material emerging and everting at the tip 4806 with the center pieces moving outward. In (2)(A), the ETT 3000 can be advanced from the base, continue through (2)(B), and translate inside the overtube in (2)(C). The ETT can be fixed at four points 4804 relative to the overtube. The fixed points can limit the forward and reverse travel of the ETT 3000. On the opposite end from 4806, the material can invert along the body, as shown in (2)(B). As shown in (2)(B) and (2)(C), the external material of the overtube can be static with respect to the environment.

In some embodiments, the introducer serves as a primary user interfaces in operating an Endotracheal tube (ETT). In some embodiments, the introducer delivers the tracheal access mechanism (TAM) and the ETT to correct position and orientation in the patient's anatomy, to enable soft robotic intubation and/or serve as a hub for the deployment of other sub-assemblies.

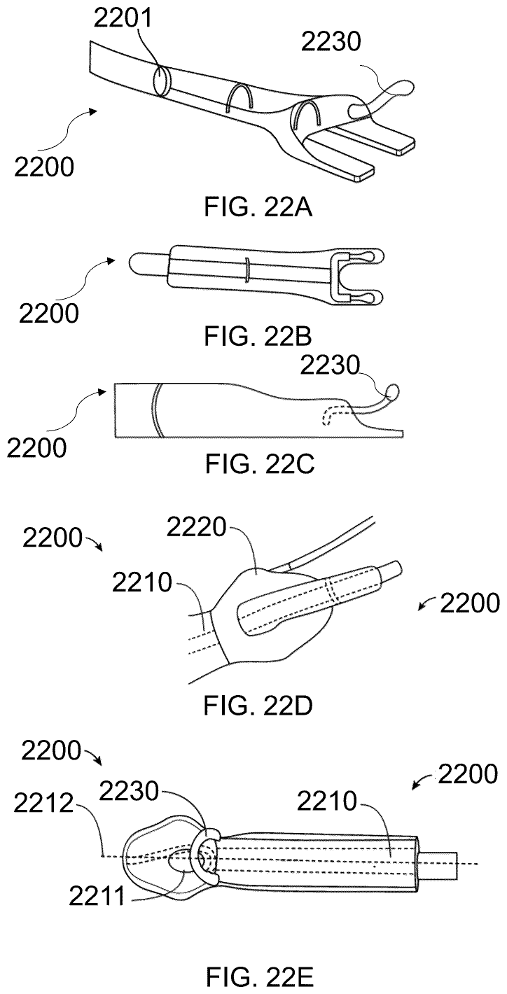

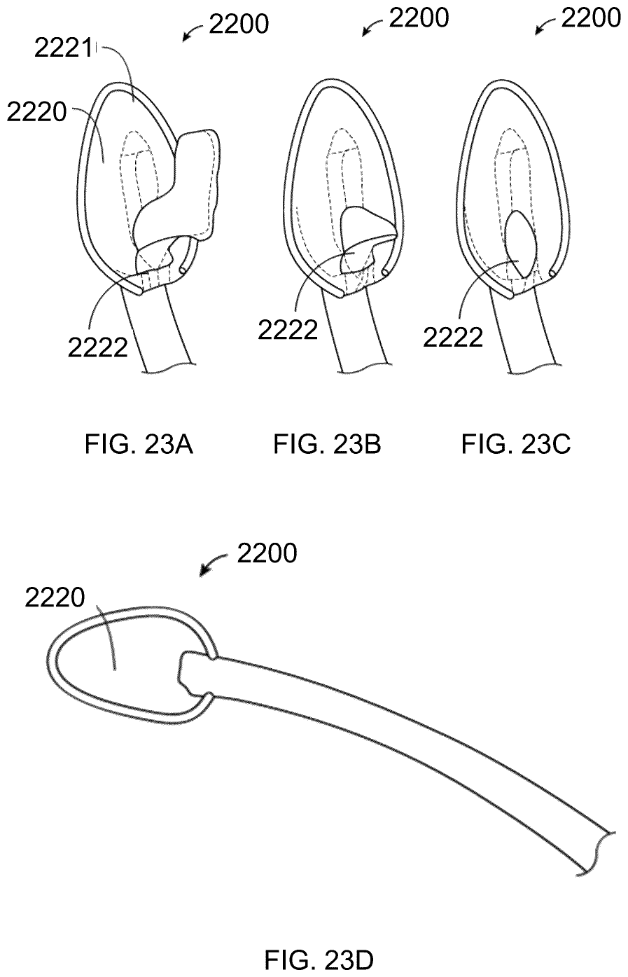

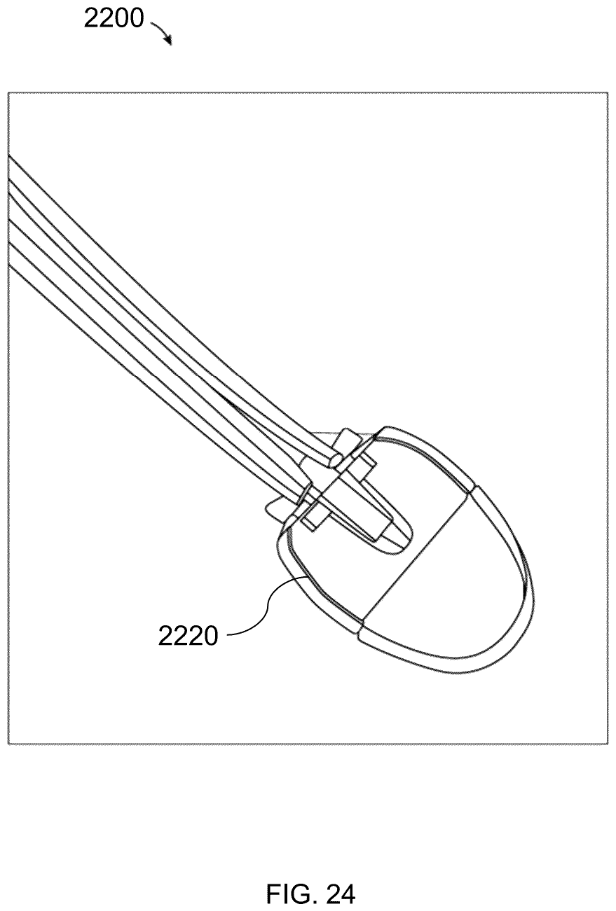

FIGS. 22A-24 show exemplary introducers for delivering a TAM. In some embodiments, the introducer may be configured to deliver an anterior TAM (aTAM) (FIGS. 22A and 25-43). In some embodiments, the introducer may be configured to deliver a posterior TAM (pTAM) (FIGS. 22D and 23-24). In some embodiments, per FIGS. 22D-24, the introducer 2200 comprises a tubular body 2210 and an ovular surface 2220. In some embodiments, the tubular body 2210 is configured to receive the tracheal intubation device. In some embodiments, a distal end 2211 of the tubular body 2210 is angled with respect to a tubular axis or elongate axis 2212 of the tubular body 2210. In some embodiments, the ovular surface 2220 is coplanar to the distal end 2211 of the tubular body 2210 and extends outwards from the tubular axis or elongate axis 2212. In some embodiments, an outer rim 2221 of the ovular surface 2220 has a greater thickness than the remainder of the ovular surface 2220. In some cases, the introducer an comprise an AShim 2230. FIG. 22A shows a perspective view, FIG. 22B shows a top view, and FIG. 22C shows a side view of a soft flexible unibody introducer with an open channel in the back to separate the ETT. FIG. 22D shows a top view of a trap door embedded in the unibody. FIG. 22E shows a top view of an AShim 2230 embedded in a unibody. In some embodiments, the ovular surface 2220 comprises a flap 2222 configured to, in a collapsed state per FIG. 23C, cover at least a portion of the distal end 2211 of the tubular body 2210. In some embodiments, the flap 2222 configured to, in an expanded state per FIGS. 23A-23B, in an expanded state, extend perpendicular to the ovular surface 2220. In some embodiments, the flap 2222 configured to, in the expanded state, extend within at least 20 degrees of perpendicular from the ovular surface 2220.

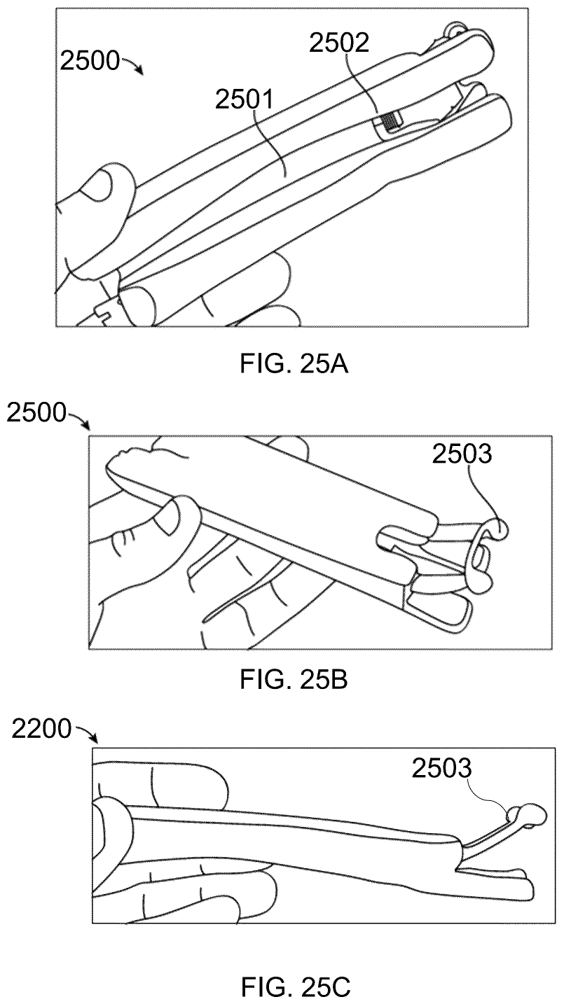

FIGS. 25A-25C show exemplary introducers 2500 for delivering an anterior TAM (aTAM). In some embodiments, Per FIG. 25A, the introducer 2500 comprises a vine channel 2501 to house the vine robot, and a vine robot attachment feature 2502. In some cases, there may be an open channel in the back to house the vine robot. The open vine channel 2501 can allow for easy separation from vine robot during removal. In some embodiments, per FIG. 25B, the introducer 2500 comprises an anterior shim 2503. The overmold can comprise a soft, flexible elastomer.

In some embodiments, the introducers 2500 herein employ tissue interaction to properly advance the TAM to the hypopharynx. In some embodiments, the introducers 2500 herein include a continuous connection between the TAM and the advancement feature. In some embodiments, the unibody introducers 2500 herein employ manual advancement. In some embodiments, the introducers 2500 herein require fewer delivery steps. In some embodiments, the introducers 2500 herein enable the ETT to be stored integrally or detachably.

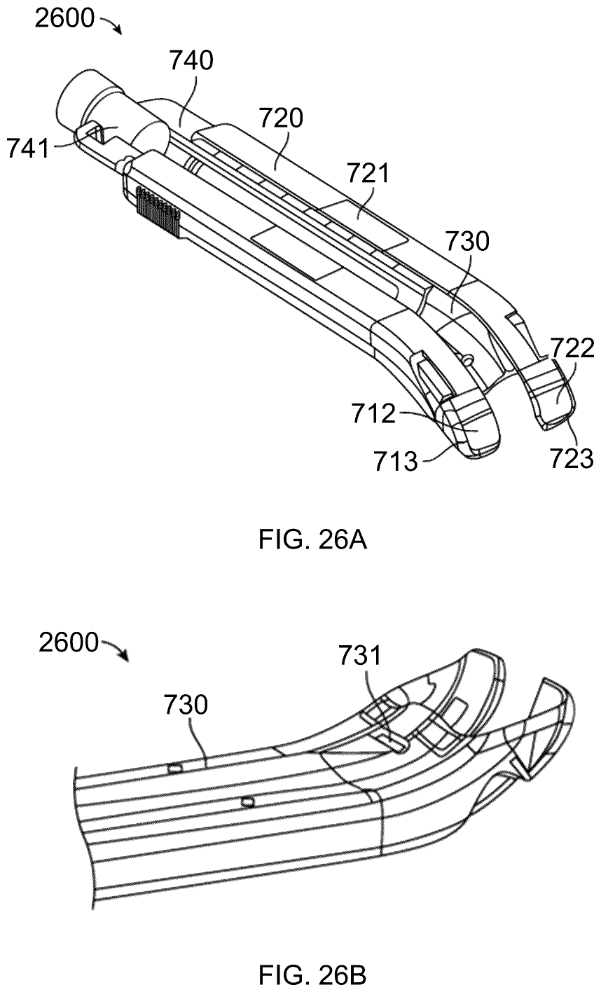

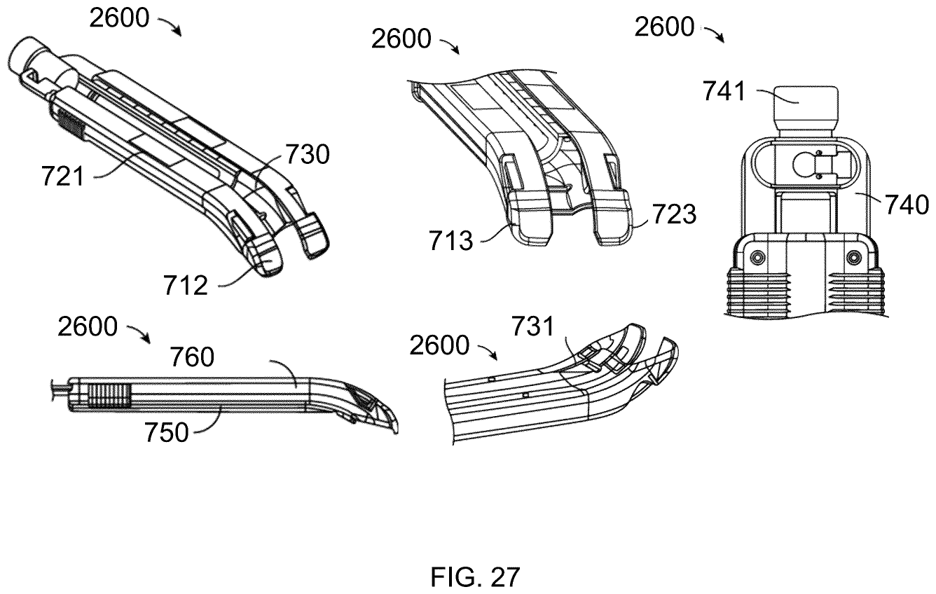

FIGS. 26A-27 show an exemplary multibody introducer 2600 configured to deliver an aTAM with various anatomical datum features. As shown, the introducer 2600 comprises a first datum 712 and 722 which stops against the oropharynx of the patient. As shown, the introducer 2600 comprises a second datum 721 that, when inserted into the mouth of the patient, rests to seat against their incisors to limit device tilt. A distance between a first point 713 and a second point 723 of the introducer 2600 can self-center into the oropharynx as it slopes posteriorly. In some embodiments, the first point 713 and the second point 723 are rounded to reduce resistance against tissue folds. In some embodiments, per FIG. 26A, the introducer 2600 comprises an anterior slot 730 configured to separate from the vine robot while preventing compression the uvula when placed in the back of the patient's oropharynx. Per FIGS. 26B and 27, a third datum 731 can contact the tongue of the patient while minimizing tongue entrainment as the introducer 2600 is passed through the oral cavity.

In some embodiments, once the introducer 2600 is appropriately oriented in the patient's oral cavity the practitioner may deploy the tracheal access mechanism by translating a plunger 740 distally towards the patient. In some embodiments, the plunger 740 and the anterior slot 730 are configured to interact and slide smoothly relatively to each other. In some embodiments, the introducer 2600 comprises a plunger disconnect 741 that removable couples to the plunger by, for example, a snap fit or a clip.

In some embodiments, upon completion of intubation the introducer 2600 may be removed from the patient, leaving the endotracheal tube in place. In some embodiments, per FIG. 27, the introducer comprises a retraction feature 741 configured to prevent tissue snags and damage. In some embodiments, per FIG. 27, the introducer 2600 is formed of a first part 750 and a second part 760 that are adjoined.

### Tissue Management Features

Tongues can vary in volume, durometer, lubricity and topology. The variation in these factors can make the anterior surface of the oropharynx difficult for strafing the AShim and TAM against. Once the TAM is seated in the airway against appropriate anatomical structures, the epiglottis and arytenoids may be managed to maintain a patent growth channel.

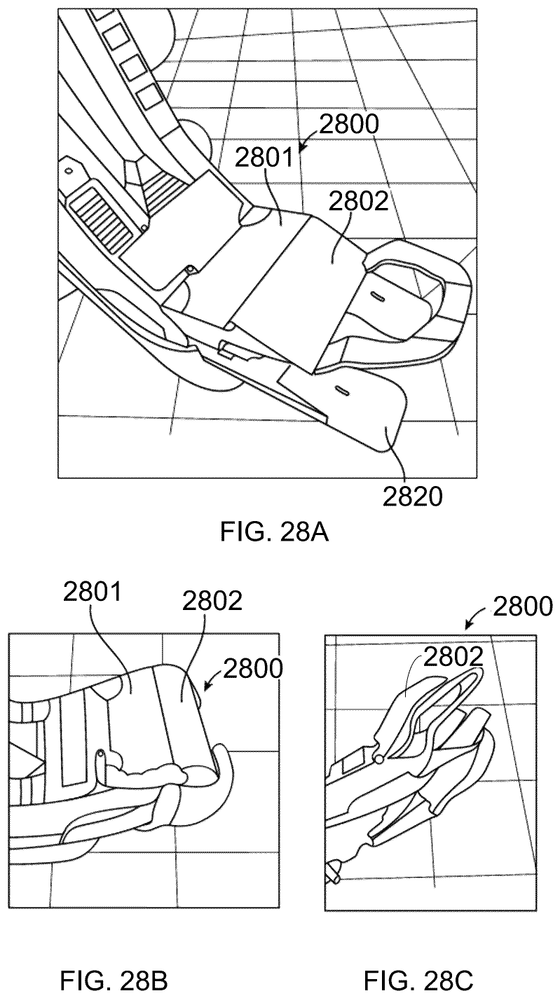

In some embodiments, per the perspective views in FIGS. 28A-28C, the introducer comprises a flap 2800 that prevents tongue entrapment throughout the operation of the ETT. The introducer flap 2802 can be a feature of the introducer to manage this tongue tissue, moving it away from the posterior surface of the pharynx as the TAM is deployed. The introducer flap can be sufficiently lengthened to allow for smooth deployment of the TAM over the tongue tissue as it transitions to anterior oropharyngeal tissue.

In some embodiments, the flap 2800 comprises a first portion 2801 and a second portion 2802 having a modulus of elasticity larger than the first portion. As such, 2802 can comprise a flexible portion whereas 2801 can comprise a rigid portion. In some embodiments, the flap can create 2800 to form a boundary surface that prevents the tongue from getting caught and pushed into the pharynx while the introducer advances to the oropharynx.

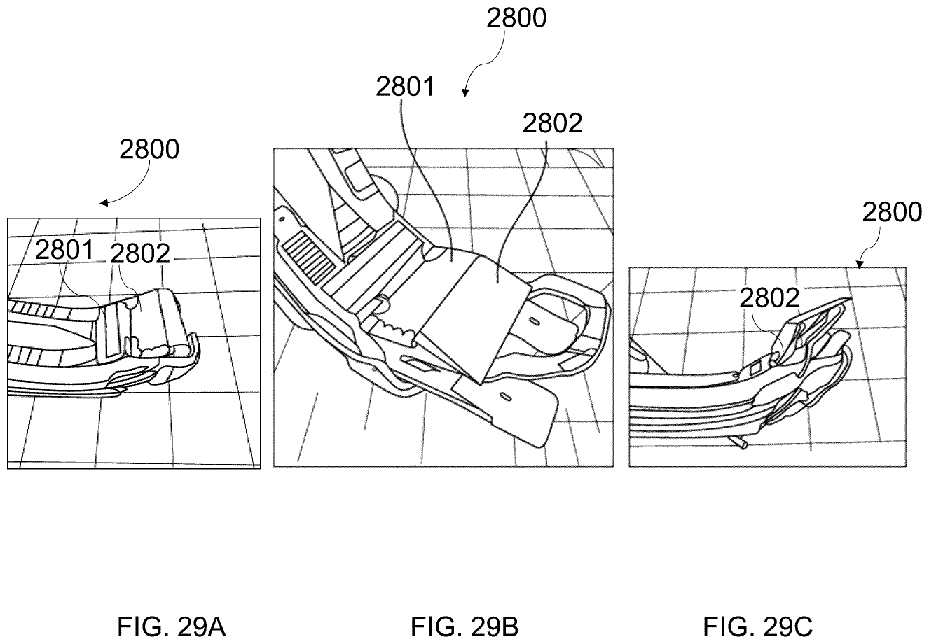

The trap door may be deployed or actuated by pneumatics, stored elastic energy or via contact with the everting vine robot. FIGS. 29A-29C show an exemplary inserter deployable flap 2800. The primary method is by contact with the everting vine robot. The vine robot may evert under pressure on the flap 2800, from a closed position in FIG. 29A, lifting the flap 2800 to an open position, per FIG. 29C, that engages the epiglottis. In some embodiments, the flap 2800 comprises a first portion 2801 and a second portion 2802, wherein the first portion 2801 is more rigid than the second portion 2802.

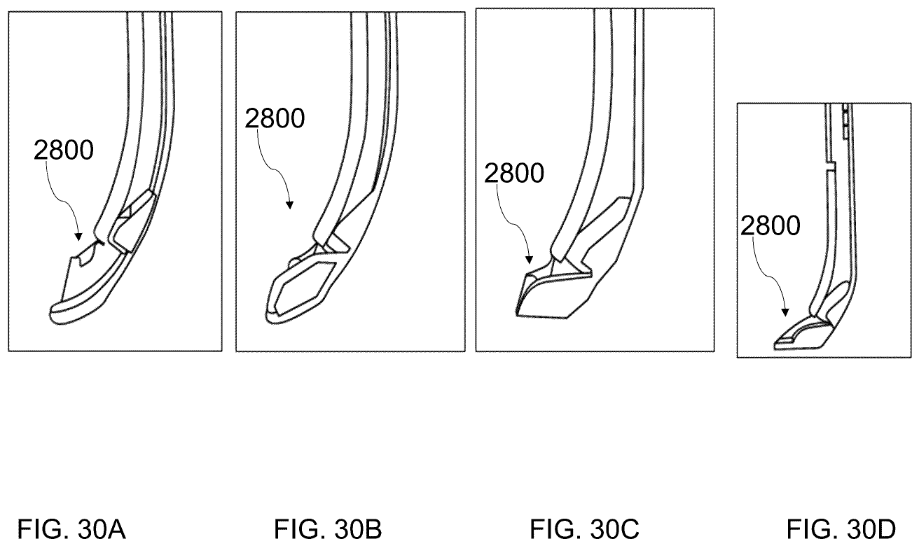

FIGS. 30A-30D show introducers with flaps with increasing contact angles. In some embodiments, if the flap has an insufficient rigidity or too great of a contact angle, displacement waves may propagation effect throughout. Such waves may push the epiglottis inferiorly, increasing the risk of the epiglottis down-folding and closing off the glottic structures. As such, the flaps herein can decrease wave propagation and increase the lifting force at its distal tip of the trap door. The rigid flap may be made of a metal, a plastic, an elastomer, or any combination thereof.

Another area that can benefit from a datum against the tissue is via the upper esophageal sphincter/cricopharyngeus, which is posterior to the previously identified anatomy exploited via the aTAM. In this approach, pTAM devices can slide behind the epiglottis, arytenoids, and larynx to seat in the hypopharynx. Similar features such as those required for aTAMs may be incorporated into pTAMs with appropriate modifications to address the different datuming mechanisms.

### Tracheal Access Mechanisms

In some embodiments, the systems herein employ a tracheal access mechanism (TAM) to actively manipulate the state of tissue in the airway, maintain a patent growth channel for intubation, and place the vine robot within a set distance from the vocal cords. Tracheal access can be produced via airway tissue management feature provide an anatomical stop that resists motion at particular points along the airway. The TAM may be translated manually by the user, autonomously translated under power, or both. In some embodiments, the TAM strafes the pharyngeal wall as it advances into the airway without guidance by visualization. In some embodiments, the TAM herein comprise posterior and/or anterior contacting components that contact the arresting structures of the anatomy and are designed to avoid entrainment of the tongue. In some embodiments, the posterior and/or anterior contacting components are arrested upon contact with anatomical features, including but not limited to, the vallecula, the glottis, the hypopharynx, the cricopharyngeus, and the upper esophageal sphincter.

In some embodiments, an anterior TAM (aTAM) provided herein comprises of a posterior shim (PShim) and anterior shim (AShim). In some embodiments, the aTAM is advanced by a plunger of an introducer. In some embodiments, the aTAM is configured to guarantee proper strafing of the pharyngeal wall throughout its deployment.

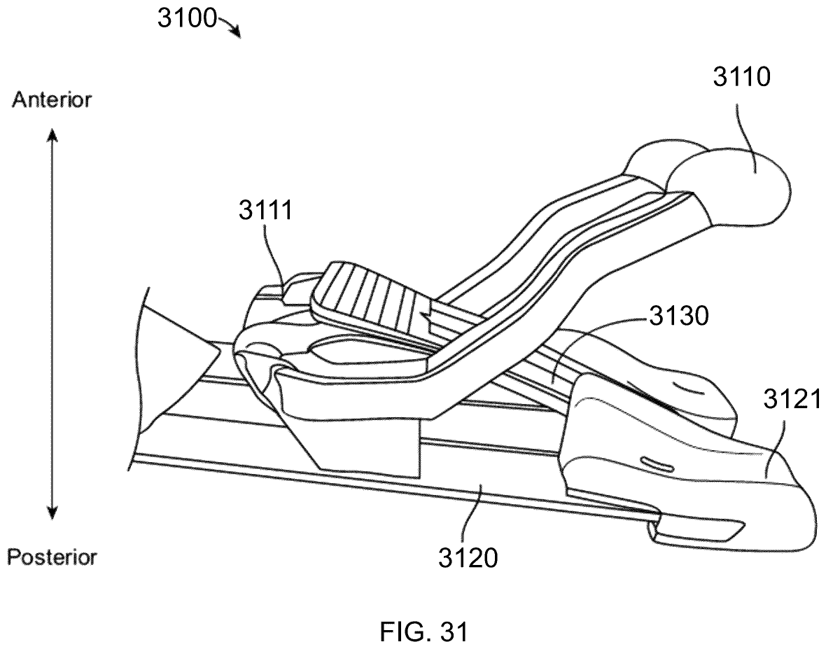

In some embodiments, per FIG. 31, the PShim 3120 of the aTAM 3100 comprises a flexible tip 3121 to provide constant force against the posterior pharyngeal tissue as the AShim 3110 strafes the anterior tissue. In some embodiments, the flexible tips 3121 stabilize the aTAM 3100 during deployment. In some embodiments, the flexible tips 3121 are configured to provide a lubricious and safe contacting surface to strafe the posterior tissue with minimal resistance from the tissue, especially in cases wherein, for example, curvature of the cervical spine varies naturally in healthy physiology and may be exaggerated due to pathological causes (e.g., kyphosis). The flexible tips 3121 may be shaped to follow the curvature of the cervical spine, improving placement of the aTAM 3100 in the preponderance of human anatomies.

In some embodiments, the AShim 3110 datums against a combination of tissues for placement of the aTAM 3100 in a vallecula state or a glottic state. In the vallecula state, the AShim 3110 can datum against the vallecula broadly, including but not limited to the hyoepiglottic ligament and the glossoepiglottic folds, which assists in lifting the epiglottis anteriorly when pressure is applied. The applied force profile may be self-centering. In the glottic state, the AShim may contact the cartilaginous structures of the anterior glottis, including but not limited to, the epiglottis, the pharyngoepiglottic folds, and the underlying thyroid cartilage.

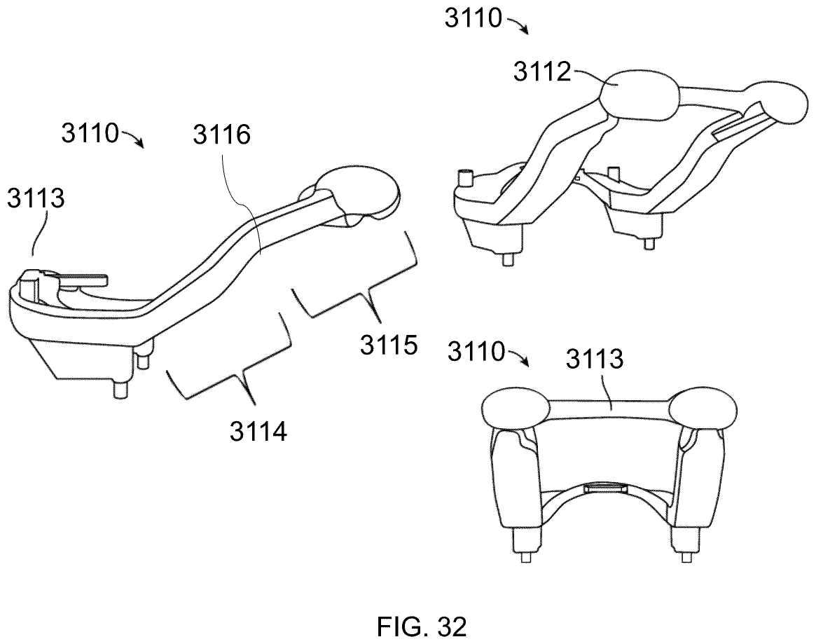

In some embodiments, per FIG. 32, the AShim 3110 comprises one or more rounded nubs 3112, a first bridge 3113 (e.g., a bumper bar), or both to establish the optimal tissue condition and increase the probability of entering the vallecula state. AShim 3110 can also comprise comprises an AShim bridge 3113, a primary segment 3114 and a secondary segment 3115 noncolinear to the primary first segment 3114. In some cases, there can be a kinked recess 3116 between first 3114 and second 3115 segments. The kinked recess can create space for the epiglottis to be anteriorly constrained.

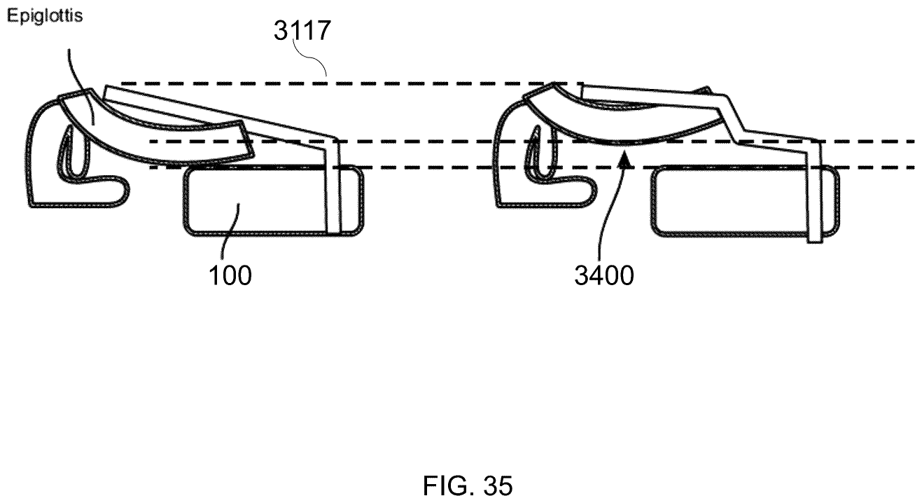

In some embodiments, per FIG. 35, the noncolinear primary first segment 3114 and secondary first segment 3115 (e.g., kinked), right, provides a vine robot growth cavity 3400 not formed when the primary first segment 3114 and the secondary first segment 3115 are colinear (e.g., not kinked), left. This can provide increased space to grow under the epiglottis. Regardless of whether the AShim has a kink, the overall AShim height constant 3117 can remain the same.

In some embodiments, per FIGS. 31 and 33A-34B, the aTAM 3100 comprises a trap door 3130 to enable the PShim 3120 to slip past and lift the epiglottis out of the way when deploying the ETT. In some embodiments, the trap door 3130 is configured for minimal contact with the arytenoids of the patient. In some embodiments, the flexible tips 3121 of the PShim 3120 retain the trap door 3130 while the aTAM 3100 advances from the introducer. In some embodiments, the trap door 3130 deploys during ETT eversion to sandwich the epiglottis anteriorly, leaving a clear growth path of the ETT into the trachea. In some embodiments, the trap door 3130 is actively actuated by an inflating body affixed to its posterior surface.

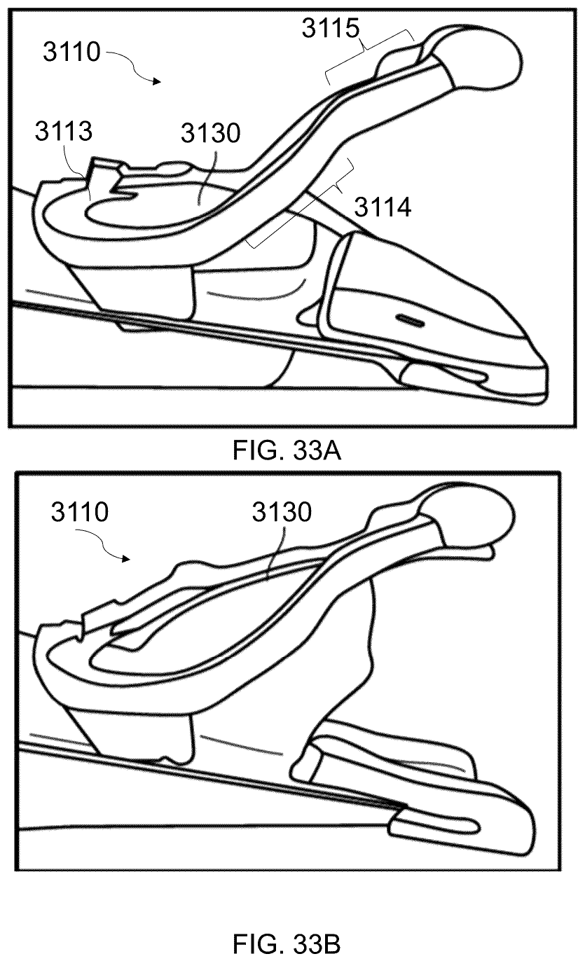

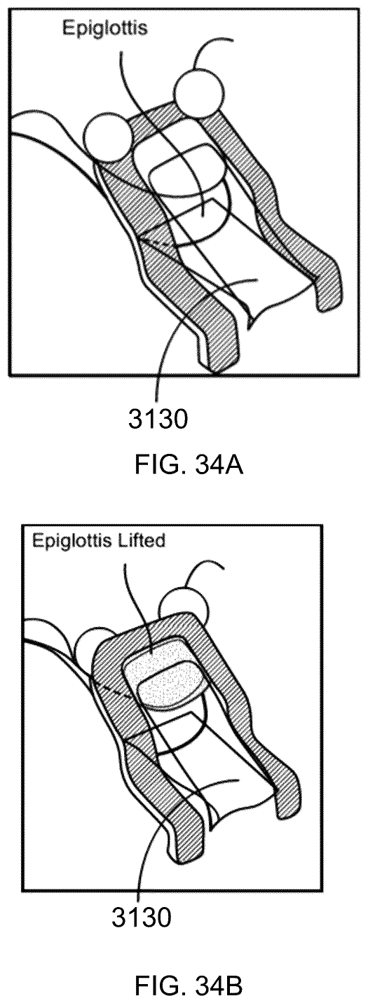

FIGS. 33A-33B show side views of a flexible trap door 3130 in a disengaged and an engaged position, respectively. In some embodiments, the trap door 3130 comprises a pin joint that pivots rather than lifts as the vine robot grows beneath. In some embodiments, in an initial, posterior position, per FIG. 34A, the trap door 3310 translates anteriorly across the pharynx, lifting the epiglottis up to the AShim 3110 where it is entrained per FIG. 34B.

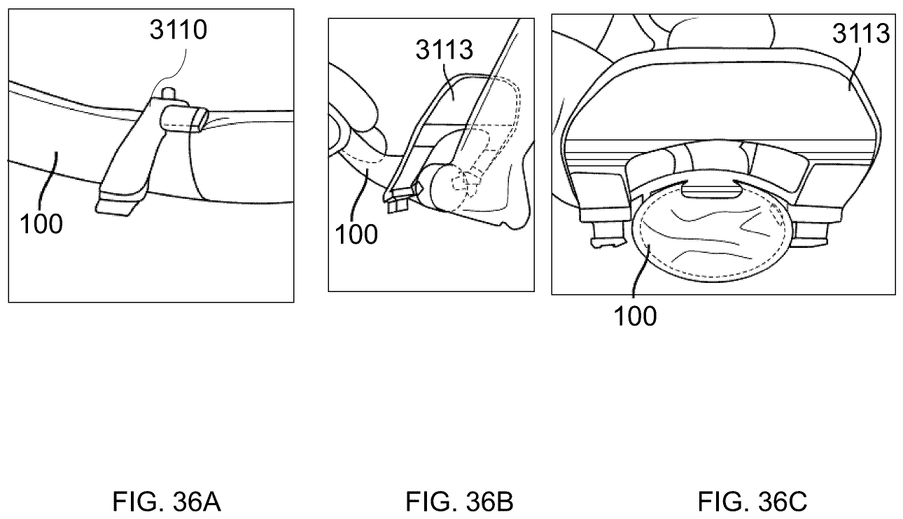

FIGS. 36A-36C show exemplary bridges 3113 of an AShim 3110. In some embodiments, the bridge 3113 secures the ETT during deployment of the TAM and facilitate detachment when the introducer is removed. Per FIG. 36A, the bridge 3113 can couple to the vine robot 100 via a one-point attachment, which offers the minimum necessary retention force to secure the vine robot 100 as it is placed by the TAM in the anatomy. Per FIG. 36B, the bridge 3113 can couple to the vine robot 100 via a two-point attachment, which can provide greater lateral stability in the deflated state while increasing the retention force in the inflated state. In some cases, per FIG. 36C, the bridge 3113 couples to the vine robot 100 via a three-point attachment with improved lateral stability.

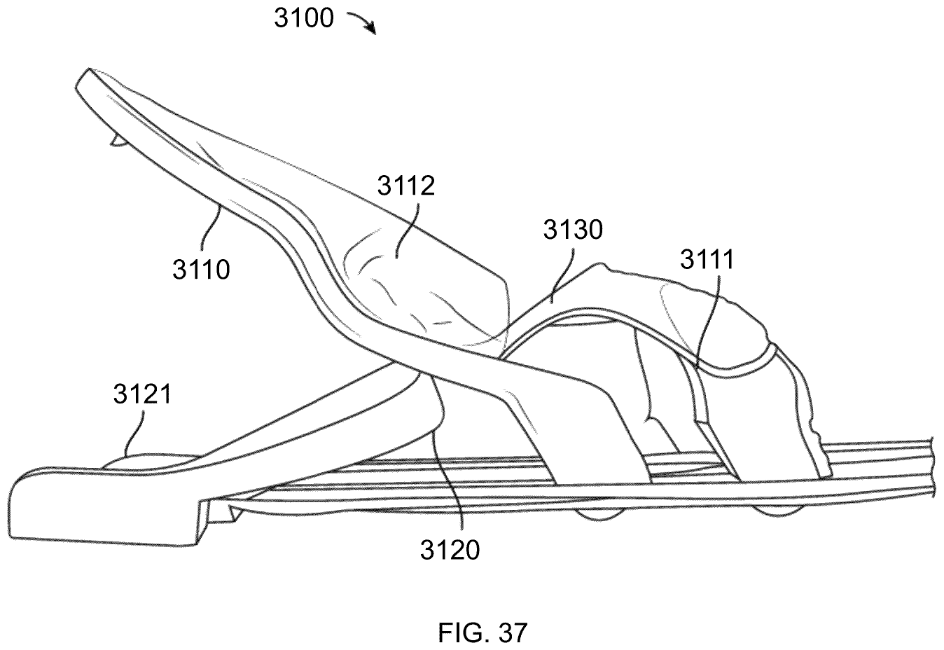

In some embodiments, per FIG. 37, the AShim 3110 comprises an AShim webbing 3112 that forms a boundary surface to constrain the growth path of the vine robot and prevent overshooting of the glottis. In some embodiments, the AShim webbing 3112 forms a volume for the vine robot to safely occupy, while allowing anterior growth of an overtube. Anterior growth can allow the vine robot to circumvent the arytenoids, which are located posteriorly.

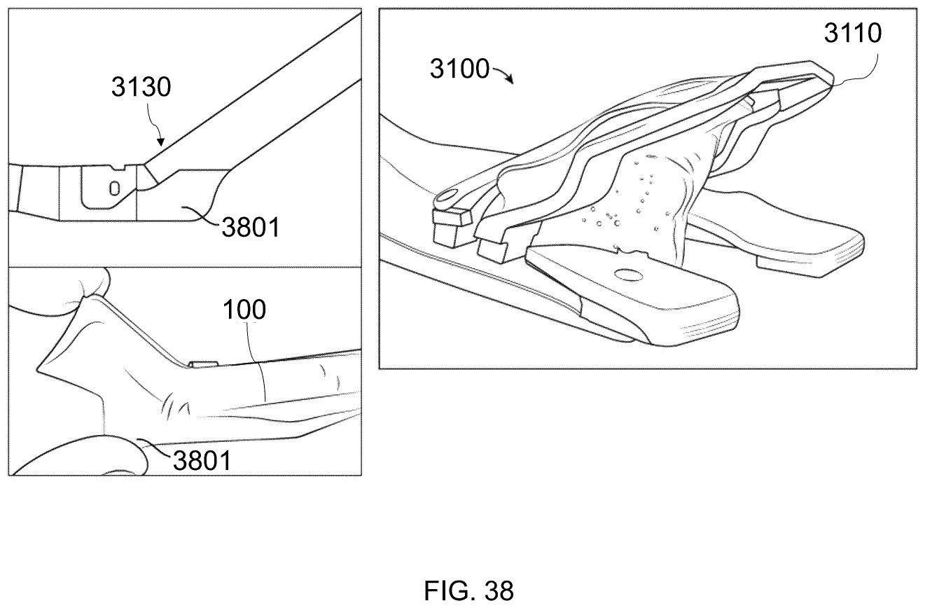

In some embodiments, per FIG. 38, as the vine robot grows anteriorly from an initial position of the AShim bridge, a nub 3801 in the vine robot 100 inflates under the trap door 3130 to deploy the trap door. In some embodiments, the nub 3801 ensures the overtube everts over the arytenoids and into the glottis.

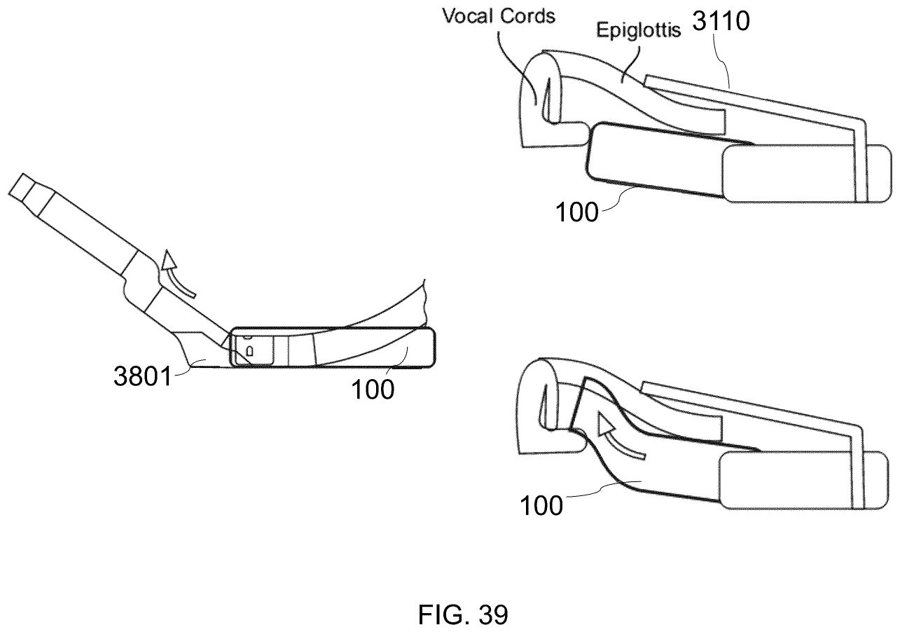

Per FIG. 39, the nub 3801 can redirect growth into the base of the epiglottis in the event that the overtube of vine robot 100 is redirected posteriorly due to tissue interactions. In some embodiments, the nub 3801 also helps to dislodge the vine overtube from the petiole in the event that the everting overtube is caught on this anatomical feature of the larynx. While the nub can provide passive direction of the vine robot 100, the vine robot 100 may additionally comprise an active (e.g., inflatable) nub. FIG. 39 also shows how the AShim 3110 rests on the epiglottis.

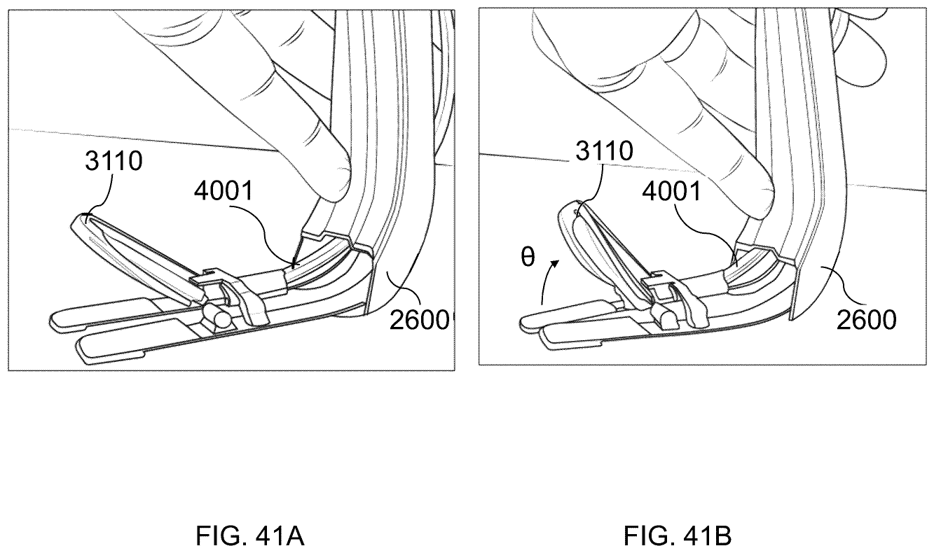

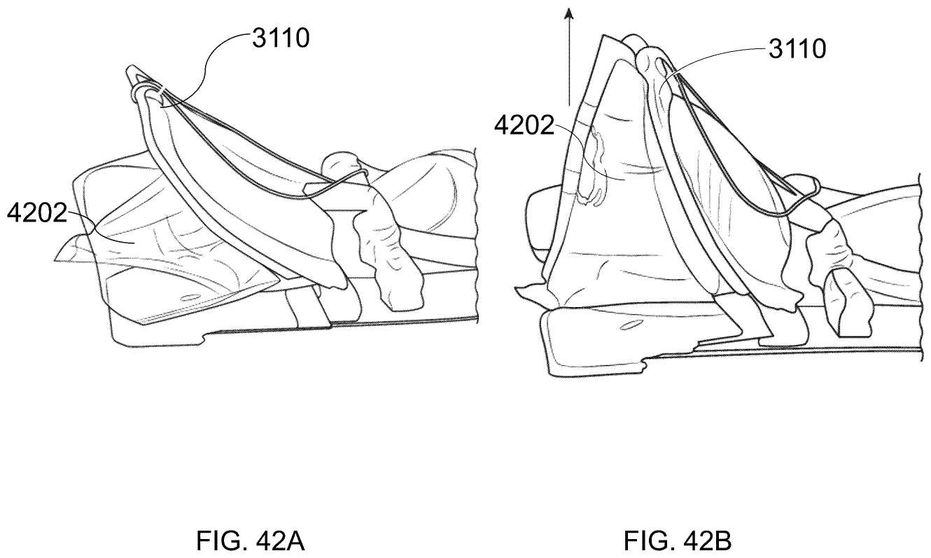

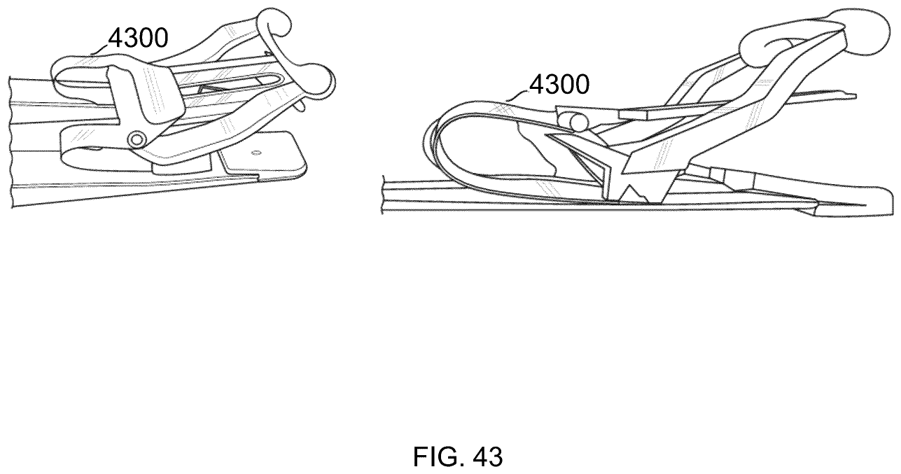

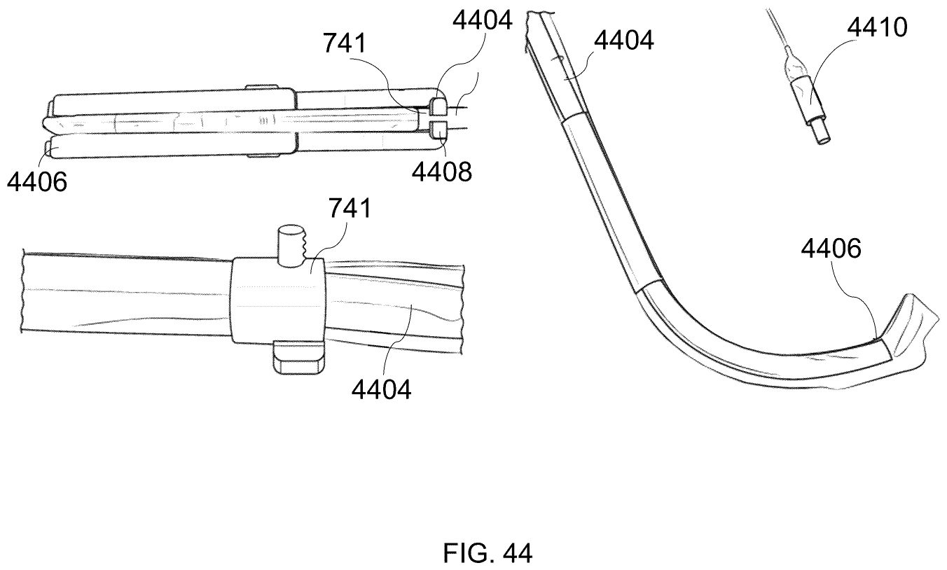

In some embodiments, the AShim is passively controlled to change the angle of the AShim by angle θ. In some embodiments, per FIGS. 41A-41B, the AShim 3110 is actively controlled via a routed cable 4001 to the tip from the base of the introducer 2600, such that tension in the pull-cable corresponds to angle θ change in the AShim. FIG. 41A shows little or no tension on the pull cable, and FIG. 41B shows tension applied to pull the cable and the cable responding by lifting. FIGS. 42A-42B show the AShim 3110 actively controlled via a gas (e.g., pneumatic inflation of a pneumatic device). The pneumatic device 4202 can be a bag-like device. FIG. 42A shows the pneumatic device deflated and the AShim at a low angle, and FIG. 42B shows the pneumatic device inflated to lift the AShim by an angle. In some embodiments, per FIG. 43, the aTAM comprise a retractor 4300 which provides geometry for the tissue to slide over, preventing snags or damage to the tissue during removal of an aTAM. In some embodiments, per FIG. 44, the plunger disconnect 741 may be attached to the plunger by either a snap fit, twist-off, clip, or other mechanical means at attachment 4408. The right figure shows a vine sheath 4404 and vine bridge attach 4406 surrounding the sheath 4404. The upper right figure shows overtube 4410.

### ETT Direction Control

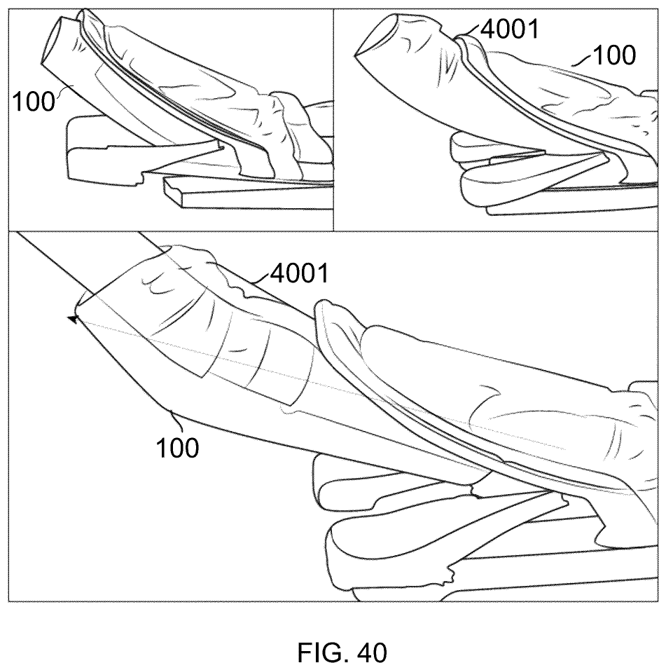

FIG. 40 shows a cable-actuated vine ETT direction control, where a cable 4001 can be routed on the anterior surface of the vine robot 100 through to the proximal base of the introducer where the user can apply tension to the cable 4001 to shorten it. In some embodiments, shortening the cable 4001 redirects the growth of the vine ETT. In some embodiments, the cable 4001 is added to a posterior surface of the vine robot 100, such that both directions may be controlled. The cable 4001 can be properly tensioned to return to the nominal direction when no additional forces are applied. The upper left figure shows how eversion begins, the upper right figure shows how the eversion angle can be controlled, and the bottom figure shows the full angle.

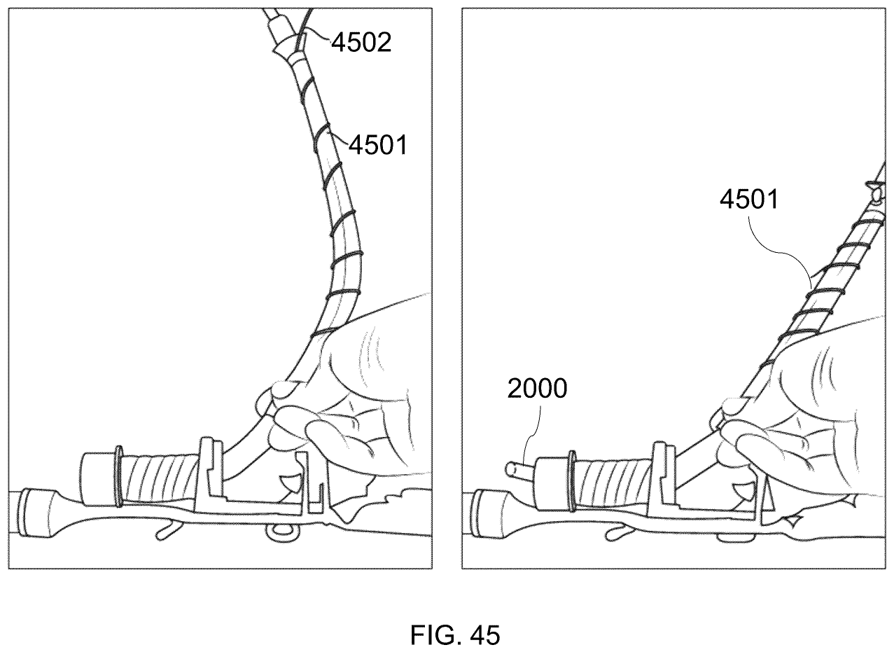

While the vine ETT can be deployed manually, FIG. 45 shows an exemplary automatic ETT deployment for increased deployment force. As shown, a spring 4501 can provide an advancement force relative to the 4502 of vine ETT 2000.

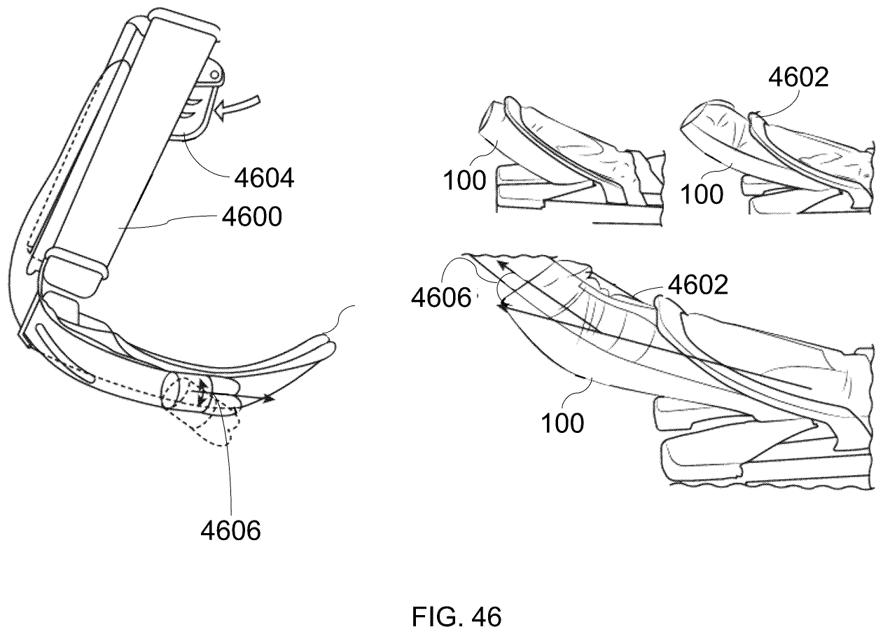

A laryngoscope 4600 with a vine robot 100 is shown in FIG. 46, which can enable visualization of the airway, manual manipulation of the tissues to create a patent airway, and placement of the vine robot in an advantageous position prior to an intubation attempt. Once the vine robot is under the epiglottis, the everting mechanism of the overtube can replace a skilled and dexterous user in placing the ETT 2000. The laryngoscope may have visualization (camera, video, fiber optics and/or mirrors). The vine robot may be manually inflated via syringe, pre-inflated, or be inflated via a pressure vessel stored in the device handle. The vine robot can grow or retract by manually translating the ETT from the proximal end. The growth angle can be manually controlled by cables 4602 that are actuated by an interface in the handle of the device.

The right three figures of FIG. 46 show an angle change with a laryngoscope introducer. The upper middle figure shows eversion beginning. The upper right figure shows that the eversion angle can be controlled, and the bottom right figure shows the full angle change θ.

Actuator 4604 of laryngoscope 4600 can be pressed. In some cases, the angle change can depend on the depth that actuator 4604 is pressed. The angle change can depend on the number of times the actuator 4604 is pressed. The angle conveyed from the actuator 4604 can determine the change in angle 4606, which can control the angle of eversion.

### Methods of Intubating a Patient

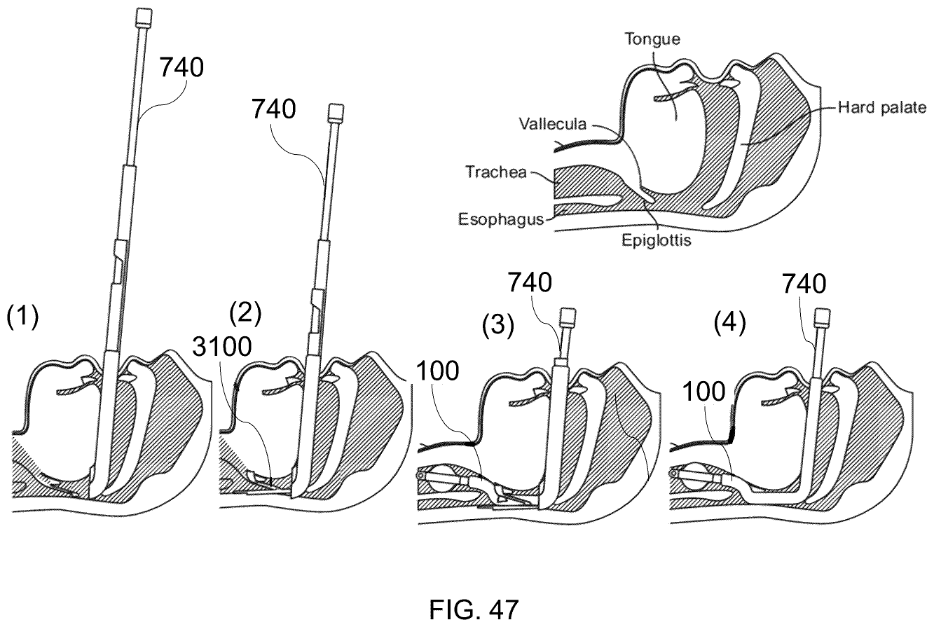

FIG. 47 shows diagrams of an exemplary method for intubating a patient. As shown, the tracheal intubation system can be (1) inserted into the mouth of the patient to the pharyngeal wall; (2) the plunger 740 can be advanced distally toward the patient to eject the ATAM 3100; (3) the primary vine robot 100 can be advanced distally between the first bridge and the posterior shim of the ATAM, (4) the ATAM can be disconnected from the primary vine robot; and the ATAM and the introducer can be removed from the patient's mouth.

### Unibody Introducers

The introducers described herein can be unibody introducers. Unibody introducers can have the advantage of requiring fewer steps to deliver but apply larger forces to the tissue by needing reaction forces to bend the unibody around the turn into the hypopharynx.

The unibody introducers can enable the vine ETT to be stored within the unibody and can be integral or detachable. Unibodies may be manufactured out of various plastics, both thermoset and thermoplastic varieties (e.g., silicones, TPUs, TPEs, etc.).

In some embodiments, unibodies may include channels through them that enable the passage of tools, cameras, and catheters. Some configurations may include an integral camera, and may connect to an external monitor, power supply, and camera driver circuit, and other electronics etc.

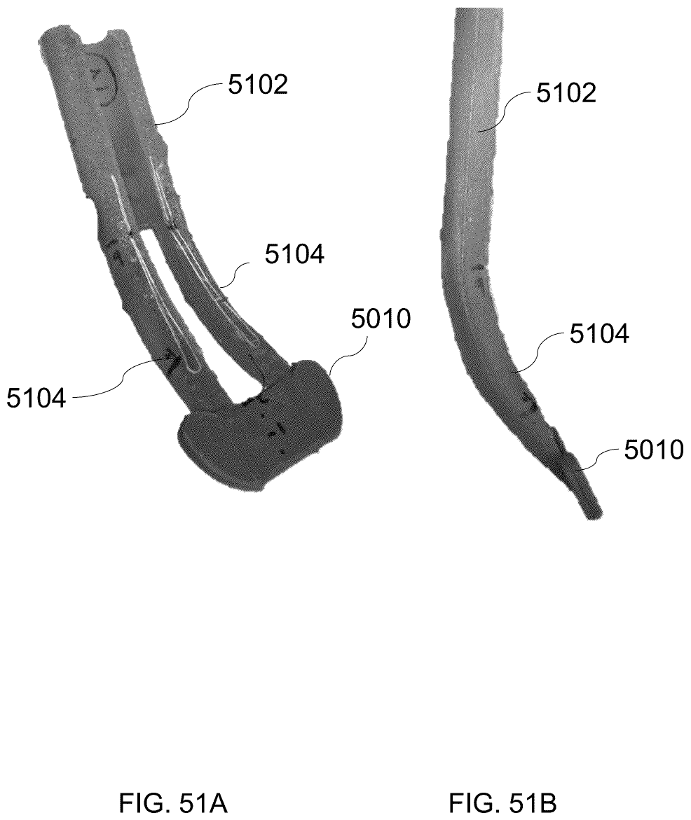

FIGS. 51A-51B show a unibody introducer that includes both connected 5102 and unconnected members 5104. In some cases, each member may define a pathway through which the ETT subassembly passes and is connected to the unibody. The unconnected members 5104 can enable the device to avoid contact with the C-spine, which may ensure proper placement of the TAM 5010 by eliminating introducer roll due to C-spine contact conditions. TAM 5010 can comprise the TAMs described above, such as TAM 3100, or other TAMs. For example, the present TAM may be a posterior TAM whereas TAM 3100 can comprise an anterior TAM. Connected members 5102 can couple to unconnected members 5104, which can couple with TAM 5010. TAM 5010 can comprise a datum, as described below. FIG. 51A shows a perspective view of the unibody introducer and FIG. 51B shows a side view of the unibody introducer. In some cases. The unconnected members 5104 may comprise openings for the compliant members described below.

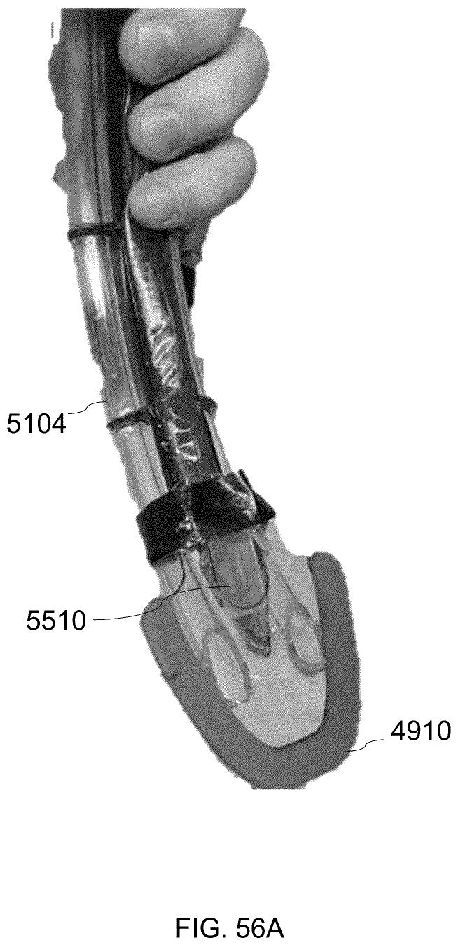

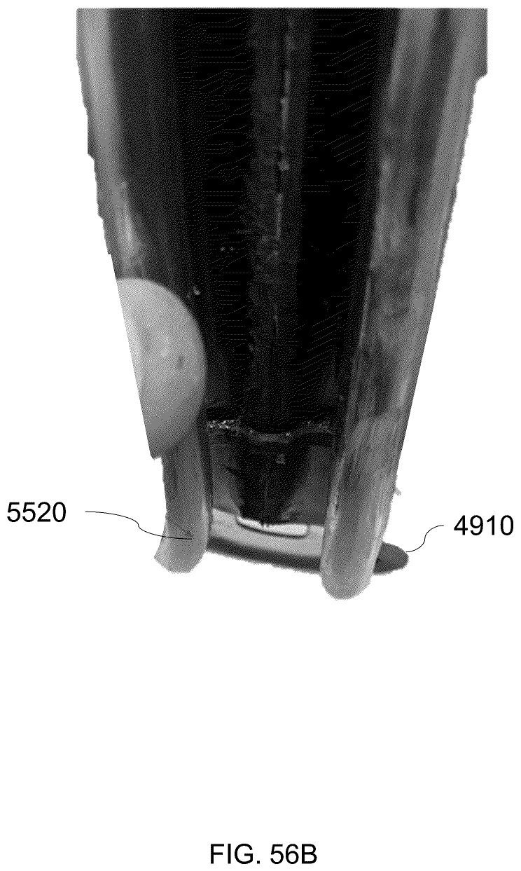

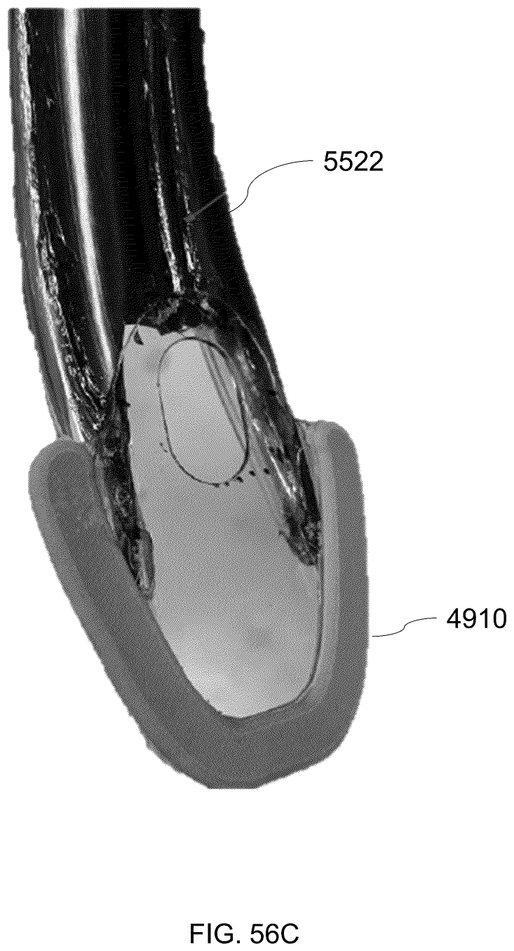

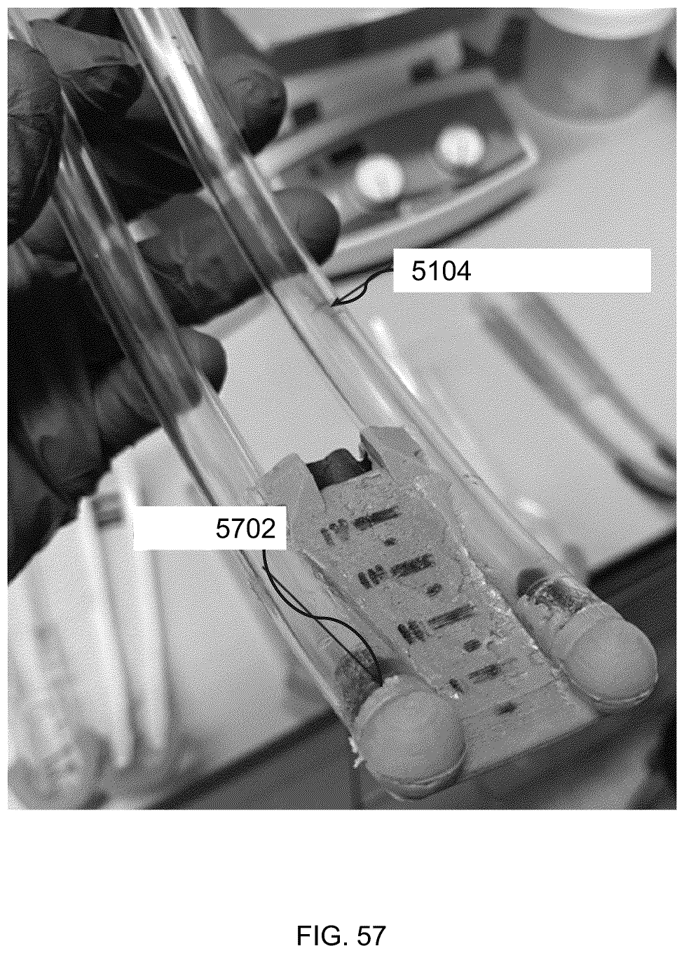

FIGS. 56A-57 show versions of a unibody introducer that include entirely unconnected members 5104. FIG. 56A shows a unibody introducer including a pair of unconnected members connected to a TAM and loaded with an ETT. FIGS. 56B-56C show a connected introducer including a pair of lateral rails configured to contact posterior tissue and preserve a volume between connected members, and a connected anterior surface configured to support a datum. As shown in the perspective view of FIG. 56A, the datum 4910 can be connected to the unconnected members 5104 through the hinge 5510. FIG. 56B shows a rear view of the datum 4910 and lateral rails for contacting posterior tissue 5520. FIG. 56C shows a perspective view with the datum 4910 and a connected anterior surface 5522 to support the datum. FIGS. 56B-56C show a connected introducer that is configured to preserve the volume between connected members. This cross section may take varied shapes but is intended to produce a midline offset from the posterior pharyngeal wall, instead of maintaining contact laterally. This may take the general shape of a “C” or “U”, with the open end in contact with the posterior pharyngeal wall. FIG. 57 shows a perspective view of unconnected members 5104 and centering pins 5702. Similar to that shown in FIG. 51, the versions of FIGS. 56A-57 can enable a straddling of the C-spine. Additionally, the leading edge of this unibody may comprise centering pins and a TAM. In an example, the TAM can surround the glottis (more information provided below) or form a traditional datum.

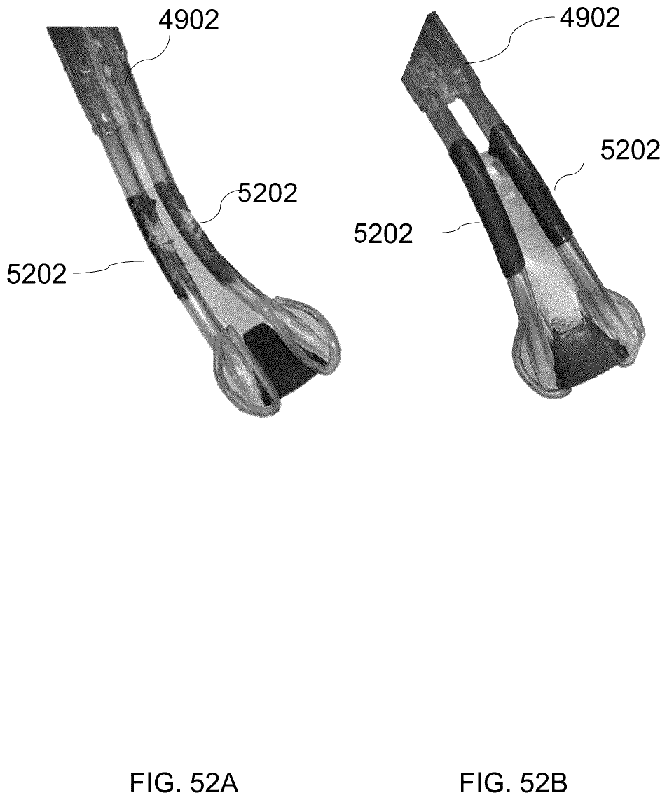

FIGS. 52A-52B show a variety of introducer 4902 that utilizes bi-directional stiffness to bend easily when in contact on the posterior surface due to compliant portion 5202, but to be rigid when in contact on the anterior surface. Introducer 4902 can comprise components from any of the introducers described herein. FIG. 52A shows a perspective front view, and FIG. 52B shows a perspective rear view of introducer 4902. The rigid anterior connector can limit posterior bending. This connector can comprise polycarbonate. The extensible posterior connector can enable anterior bending. In some cases, this connector can comprise sawtooth cuts covered in rubber.

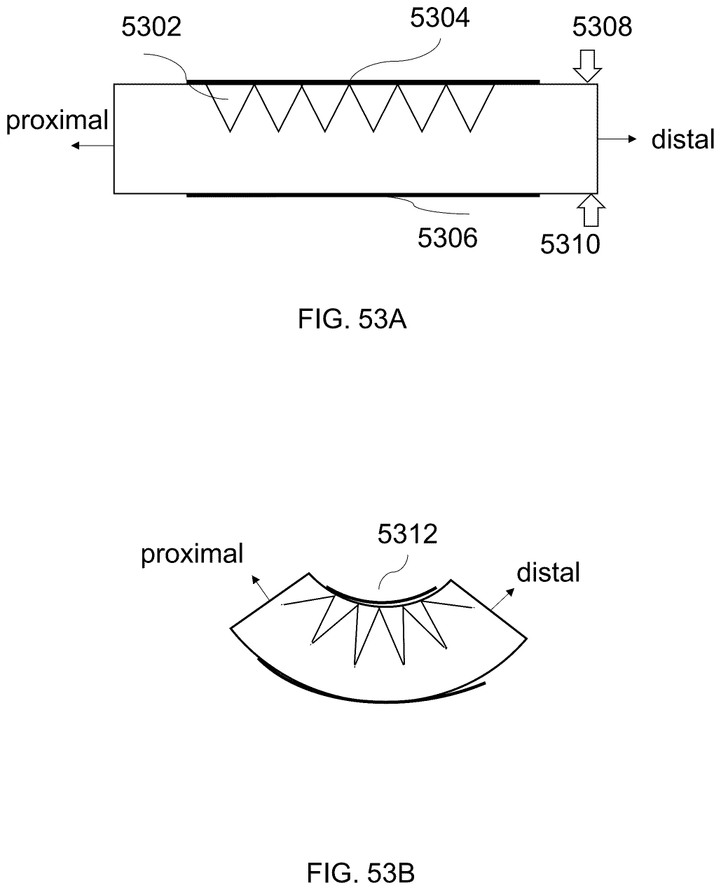

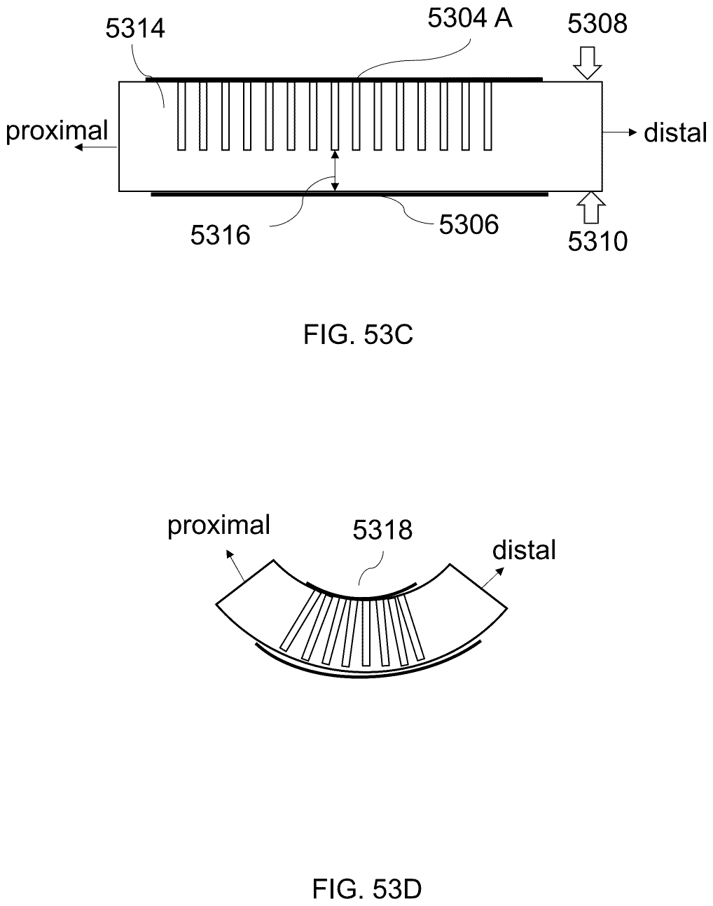

FIGS. 53A-53D show an introducer including a directionally-compliant portion including a series of groves (FIGS. 53A-53B) or pillars (FIGS. 53C-53D) configured to enhance bending in a preferential direction. In some implementations, the directionally-compliant portion includes a cover A and a cover B. In an example, the cover A or cover B may resist or aid in bending. In an example, the cover A or cover B may wrap around the introducer. FIG. 53A shows first cover (A) 5304, second cover (B) 5306, grooves 5302, first force (A) 5308, and second force (B) 5310. The left arrow can represent the proximal direction and the right arrow can represent the distal direction. The two forces can be forces pressing the first and second cover towards one another. FIG. 53B shows an arched proximal and distal direction along with bend 5312. In some cases, in the bent configuration, there may be a gap between the grooves and cover B before bending but not after. In some cases, the gap may remain.

FIG. 53C shows first cover (A) 5304, second cover (B) 5306, pillars 5314, gap 5316, first force (A) 5308, and second force (B) 5310. The left arrow can represent the proximal direction and the right arrow can represent the distal direction. The two forces can be forces pressing the first and second cover towards one another. FIG. 53D shows an arched proximal and distal direction along with bend 5318. In some cases, in the bent configuration, there may be a gap 5316 between the pillars and cover B before bending but not after. In some cases, the gap may remain.

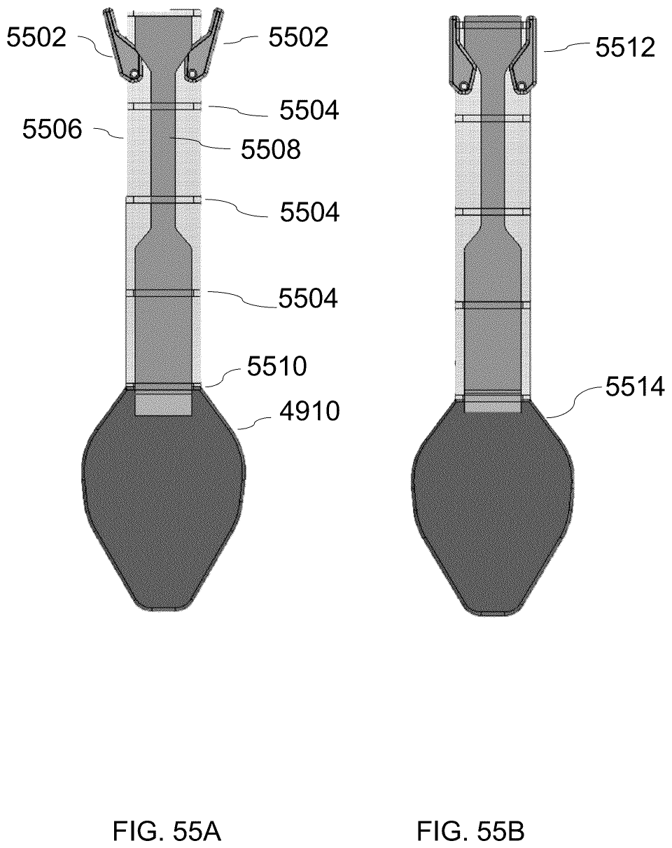

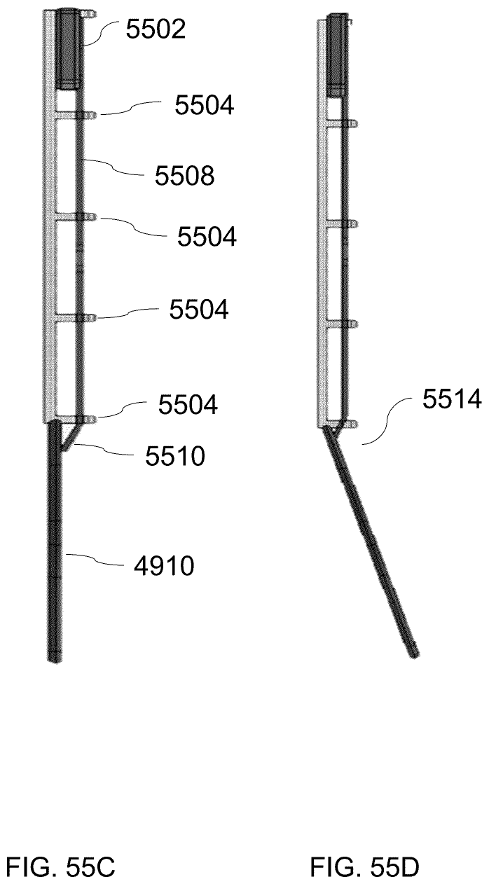

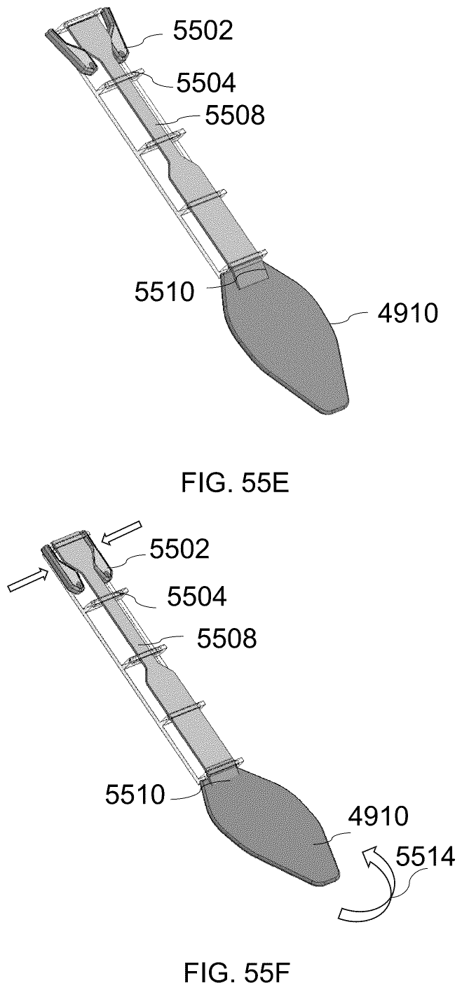

FIGS. 55A-55F show an introducer with active actuation, enabling the radius of curvature along the body of the introducer to change with applied forces. This may be implemented using a variety of techniques, but it may rely on changing length of one side of the introducer body selectively (in this case shown the “anterior” side is shortened). FIGS. 55A-55F comprise lever 5502, track 5504, base 5506, coupler 5508, hinge 5510, and datum 4910. When lever 5502 is compressed 5512, the datum 4910 can be actuated and pivot 5514. FIGS. 55A-55B show front views, FIGS. 55C-55D show side views, and FIGS. 55E-55F show perspective front views.

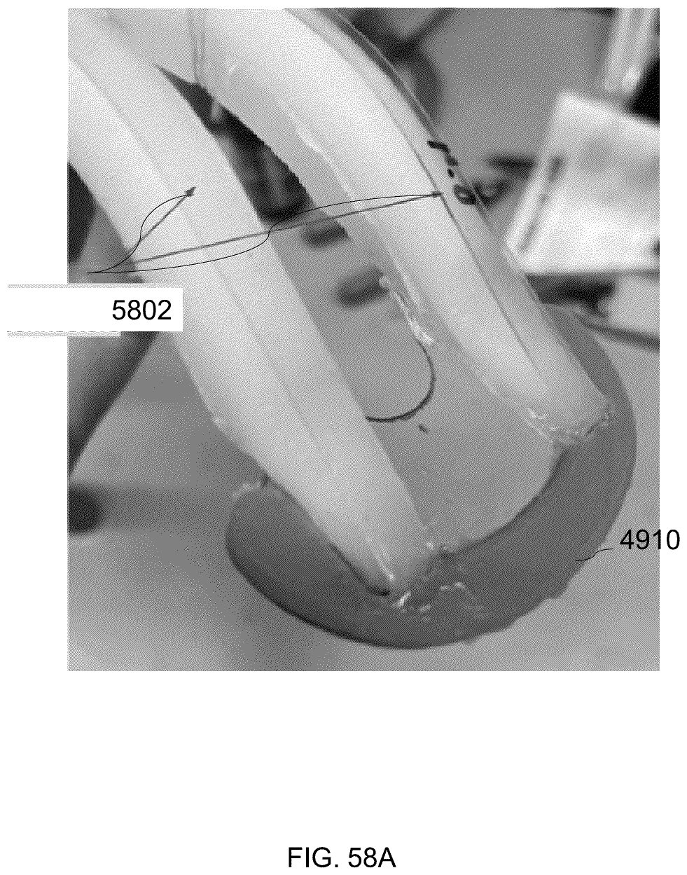

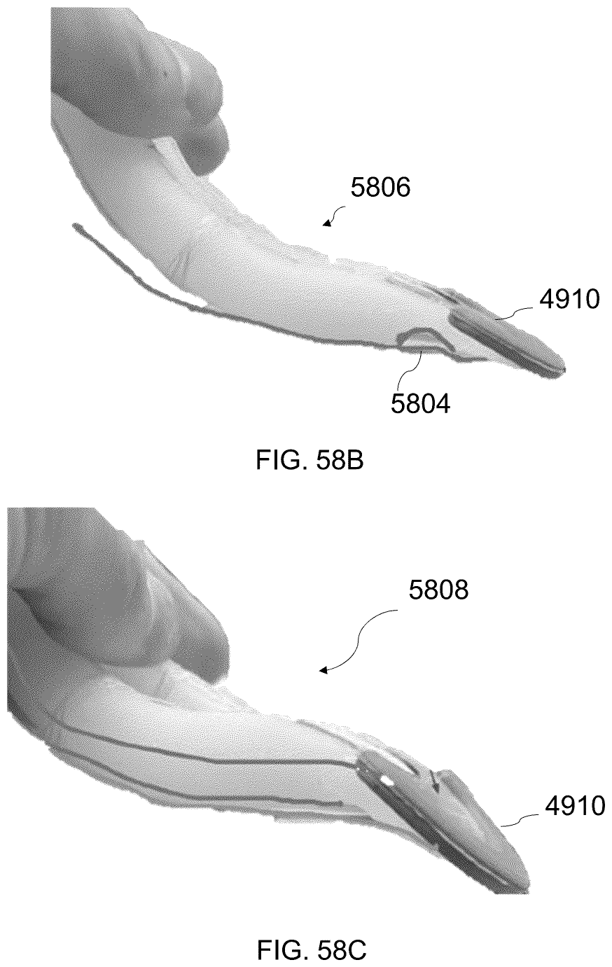

FIGS. 58A-58C show an introducer with passive actuation and a posterior valence mechanism, wherein additional curvature of the introducer body can put additional tension on the posterior side of the TAM. The posterior valence mechanism or floating connectors may induce passive actuation by augmenting curvature of a body of the introducer and cause tension on a posterior side of the TAM. The introducer can include a notch configured to form a pivot point for bending. Utilizing floating connectors, as the arc length of the posterior side increases due to bending, this strain-limited connector can pull on the posterior side of the TAM where it is connected (and is connected again to the base of the introducer). This may produce a movement in the TAM that tilts it posterior, ensuring good contact with the tissue during navigation to the hypopharynx. FIG. 58A shows a perspective view of the introducer with posterior floating connectors 5802 and datum 4910. FIGS. 58B-58C show side views of the introducer. FIG. 58B shows a nominal position 5806 of the introducer with notch 5804 and datum 4910. FIG. 58C shows an actuated position 5808 of the introducer where bending pulls the tip (e.g., tip of the introducer and the datum) in a posterior direction.

The features shown may be combined in many varieties to enable connected and unconnected members with bi-directional stiffness and varied TAM features. These introducers can also include features to defeat the epiglottis that interface with the TAM, as described in past disclosures. In some embodiments, the introducer can use appropriate geometries to round the oropharynx into the hypopharynx, and appropriate mechanical characteristics to properly interface with tissue during the navigation into the hypopharynx.

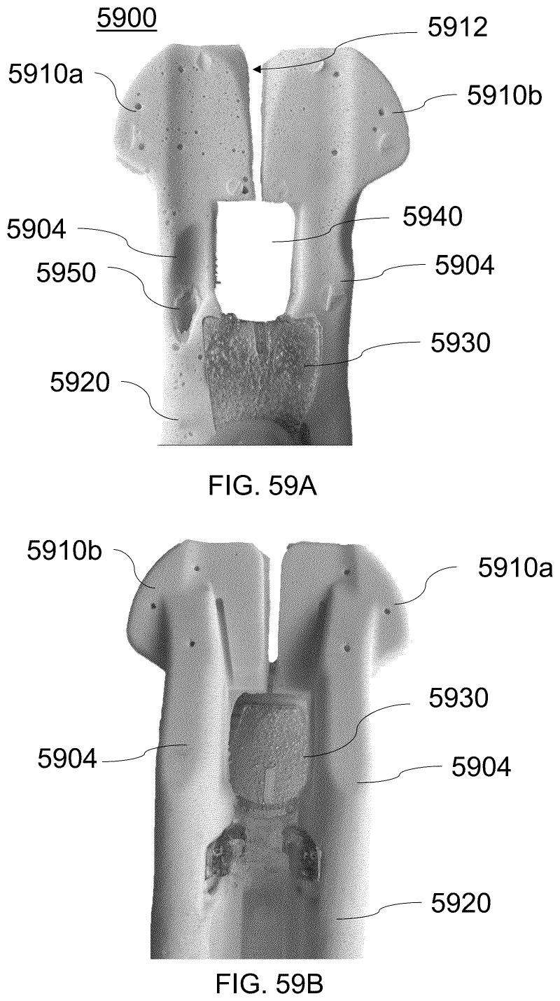

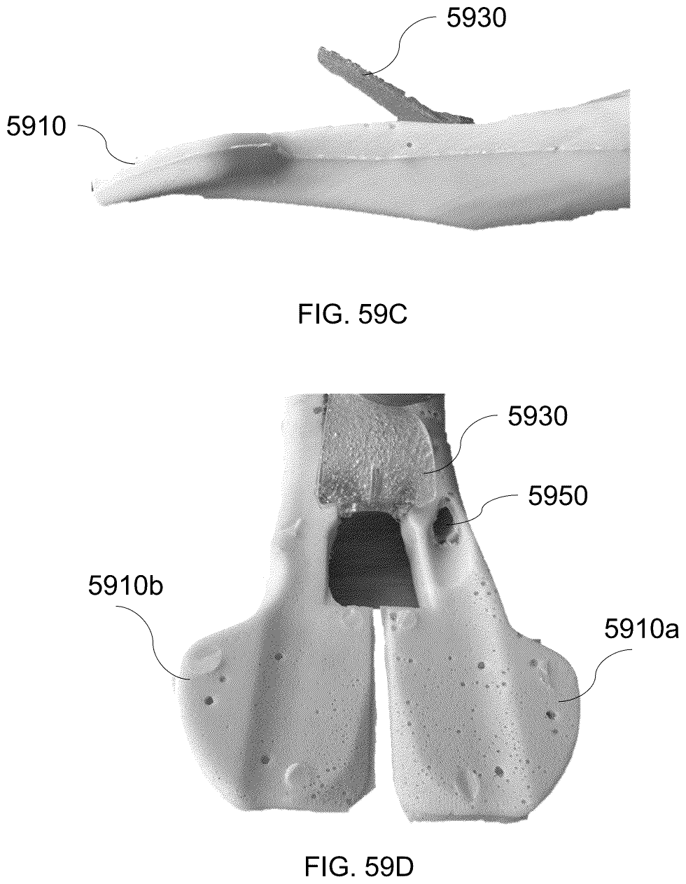

FIGS. 59A-59D show alternate views of an introducer 5900 according to an exemplary embodiment. In some implementations, the introducer 5900 may include a handle 5920, a bridge 5904 connected to the handle 5920, a TAM 5910a-b connected to the bridge 5904, a egress visualization port 5950, an epiglottic elevating element 5930. In some implementations, the bridge may include an egress 5940 for passing a ETT subassembly. In some implementations, the TAM 5910a-b may include a TAM egress 5912 through the TAM for passing the ETT subassembly. In some implementations, the TAM may be molded over the bridge forming a unibody introducer with the handle.

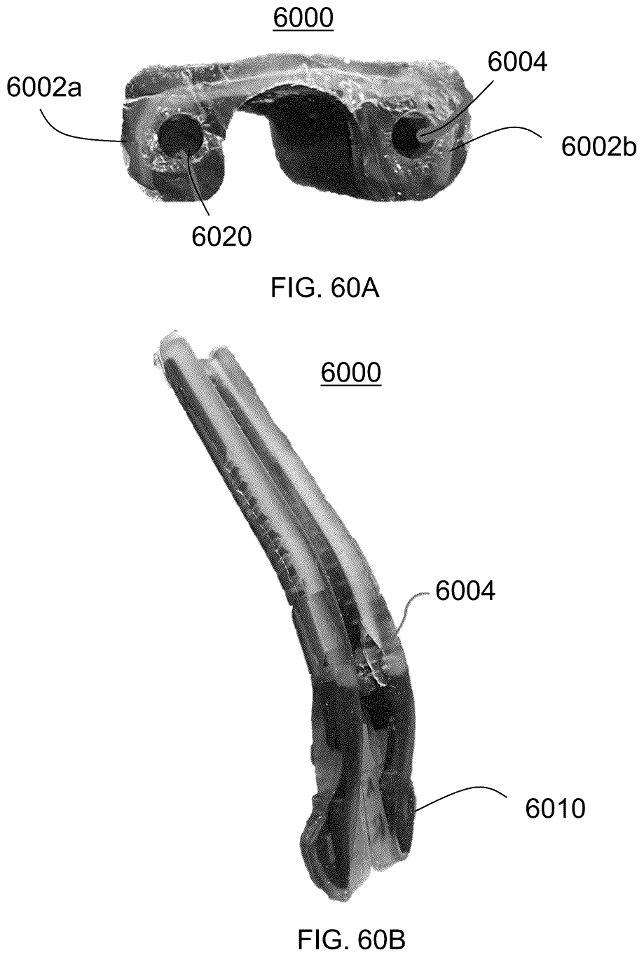

FIGS. 60A-60B show alternate views of an introducer 6000 including a gastric port 6004 according to an exemplary embodiment. In an aspect, the gastric port may be configured to allow for an orogastric tube to be passed through the introducer. In an example, the orogastric tube may be used to decompress the stomach. In some implementations, the introducer 6000 may include a visualization port 6020 along a first portion or member 6002a and the gastric port 6004 along a second portion or member 6002b. In some implementations, the introducer 6000 may include a single port for both the visualization port and the gastric port. In some implementations, the visualization port 6020 may extend through a portion of a TAM 6010. In some implementations, the gastric port 6004 may extend through the introducer 6000 but short of the TAM 6010.

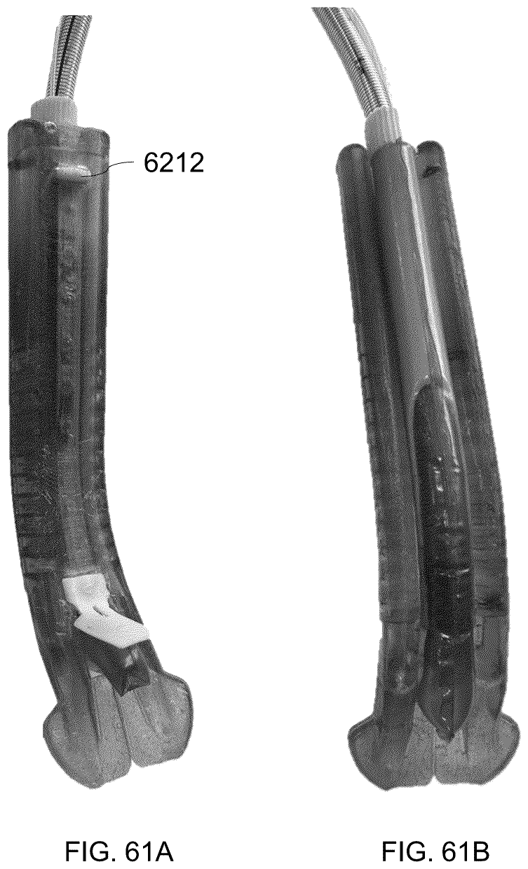

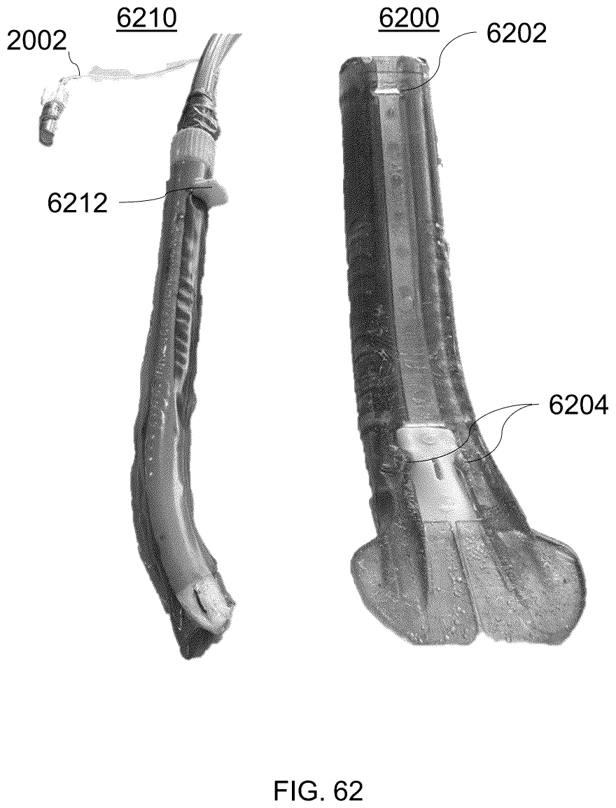

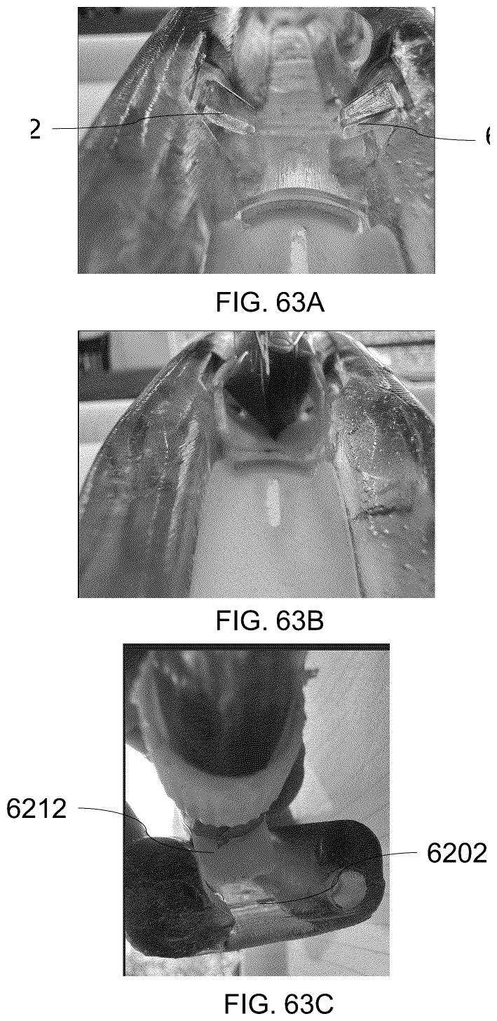

FIGS. 61A-61B show alternate views of a tracheal intubation device including an introducer 6200 with an assembled ETT subassembly 6210 to the introducer according to an exemplary embodiment. FIG. 62 shows a tracheal intubation device including an introducer having proximal 6202 and distal retention features 6204 with an ETT subassembly having a complementary proximal 6212 retention feature according to an exemplary embodiment. FIG. 63A shows distal retention features of the introducer according to an exemplary embodiment. FIG. 63B shows a ETT subassembly secured within the distal retention features of the introducer according to an exemplary embodiment. FIG. 63C shows a ETT subassembly including a proximal retention feature 6212 aligned with a proximal retention feature 6202 of the introducer according to an exemplary embodiment.

### Posterior Tracheal Access Mechanism

The purpose of the tracheal access mechanism (“TAM”) can be to exploit the anatomy in the airway to enable a patent growth channel for intubation and place the vine robot relative to the vocal cords within a known range. Tracheal access may be produced via features that manage airway tissue appropriately while simultaneously providing an anatomical stop that resists motion at particular points along the airway. The TAM may be translated manually by the user, autonomously translated under power, or by some combination. The TAM can strafe the pharyngeal wall as it advances into the airway with or without guidance by visualization. In some cases, features of the TAM that come into contact with the arresting structures of the anatomy comprise an assembly of posterior contacting components. These features can avoid entrainment of the tongue and be arrested upon contact with anatomical features, including, but not limited to the cricopharyngeus/upper esophageal sphincter (UES).

Provided herein are methods of posterior tracheal access via a posterior tracheal access mechanism (pTAM, henceforth simply referred to as TAM). This method can utilize a slim feature that stays in contact with the posterior pharyngeal wall, and slides behind the epiglottis and under the glottis, eventually coming to rest at the inferior hypopharynx upon the UES.

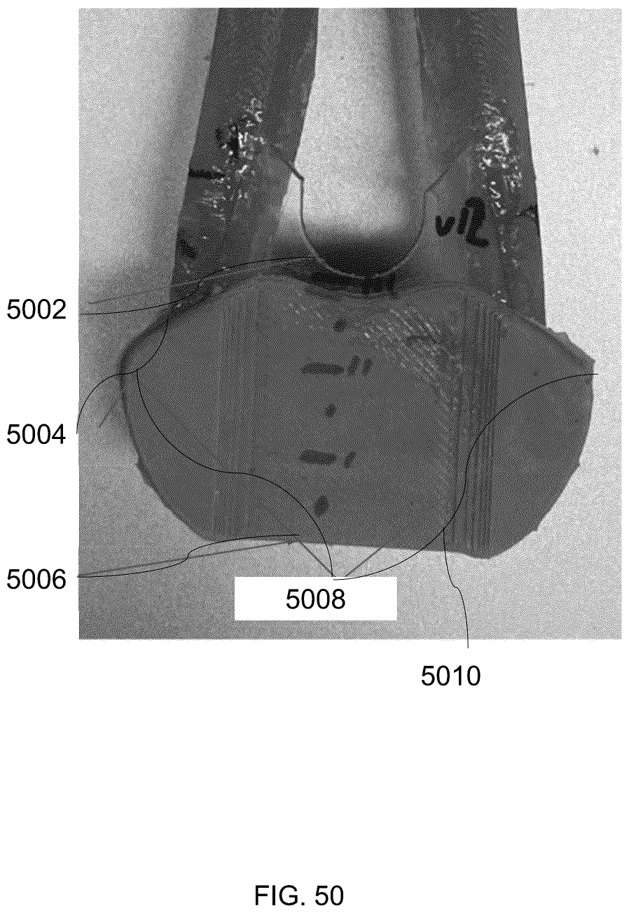

FIG. 50 shows a TAM including an ETT egress port offset and a datum having upper esophageal sphincter (UES) self-centering width and molar avoidance geometry. FIG. 50 shows a rear view of the geometry of a TAM 5010 with ETT egress port offset 5002, molar avoidance geometry 5004, UES interaction geometry 5006, and self-centering width 5008. This geometry can enable the device to effectively round the oropharynx without catching the C-spine, to enable the TAM to properly datum against the UES, and to self-center within the hypopharynx with appropriate access to the glottic opening.

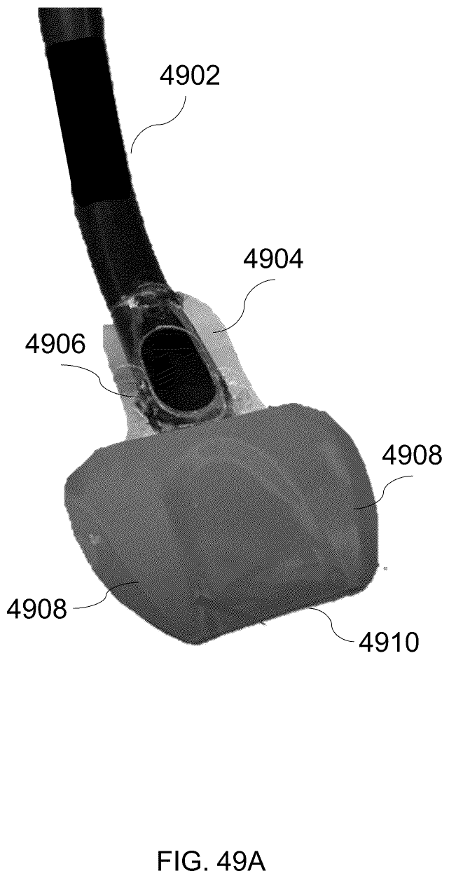

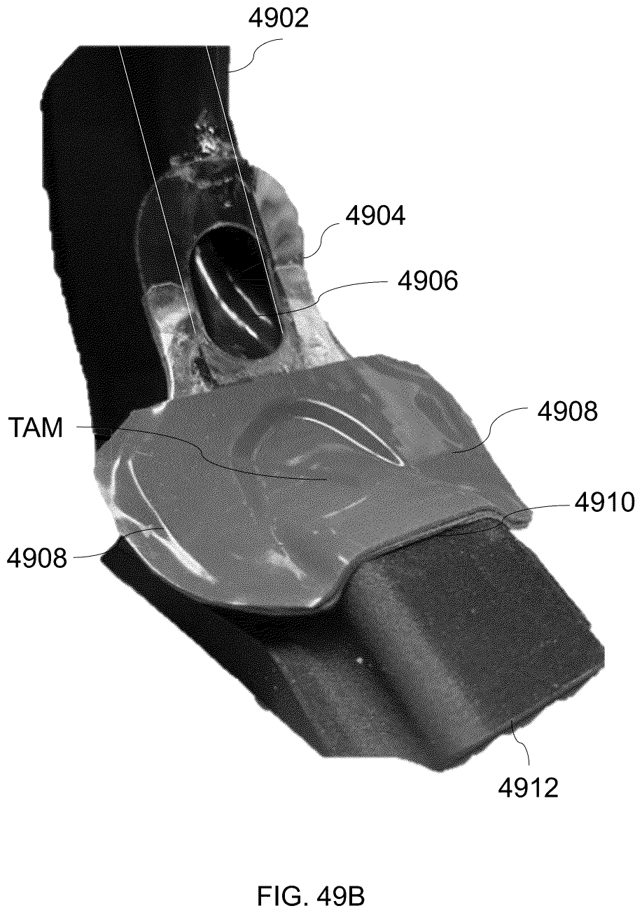

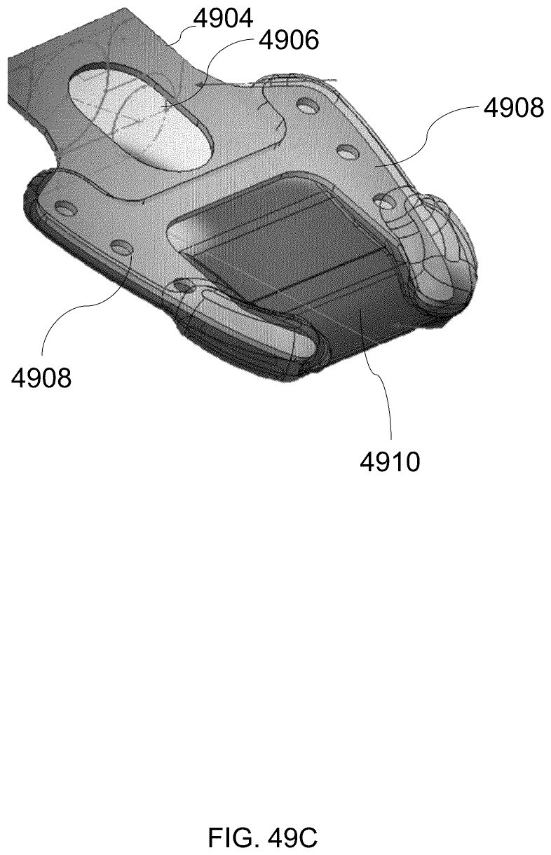

FIGS. 49A-49C show a tracheal intubation device including a unibody introducer and a tracheal access mechanism (TAM) including a base having an aperture for passing an endotracheal tube (ETT) subassembly, a bridge, and a flexible datum.

FIG. 49C shows a perspective view of how the TAM in FIG. 50 may be constructed. A distal end covering (e.g., the flexible datum) can comprise a soft material that is overmolded on top of a rigid base having a stiffer internal structure (e.g., the base). This internal structure may be modified to include an additional member embedded inside the overmolded feature, corresponding more to an “E” configuration as contrasted with the “C” configuration shown. This additional member may assist in those processes described in the preceding paragraph. The base can be configured to support a flexible datum. The flexible datum can aid in c-spine interaction. FIG. 49C shows base 4904, aperture 4906, bridge 4908, and flexible datum 4910. In some implementations, base 4904 may include a connecting portion 4905 which forms a portion of the aperture 4906. In some implementations, the base 4904 may not include the connecting portion 4905, forming an egress for the ETT.

FIG. 49A-49B show perspective views of introducer 4902 with base 4904, aperture 4906, bridge 4908, datum 4910, TAM 5010, and phantom 4912. The C-spine of a patient may be prominent, and the soft overmolded material conforms to these prominences. In some cases, the overmolded material can form a webbing to conform to varied geometries. Examples of soft overmolded material can include low durometer plastics, elastomers, silicones and the like. Rigid structural elements (e.g., made from plastic) under the overmolded material can provide self-centering and datuming.

Just as in above disclosures, tissue management features, such as a trap door, may be included in the device architectures described above to enable the device to navigate around the epiglottis and maintain a patent channel from the mouth to the trachea. The trap door may be deployed or actuated by pneumatics, stored elastic energy or via contact with the everting vine robot.

Just as in above disclosures, the ETT subassembly may benefit from some active steering. This may be done with pull tendons routed along the body to create whole-body bending of unsupported sections, or features on the ETT subassembly with discrete actuation, such as just distal to the introducer attachment point.

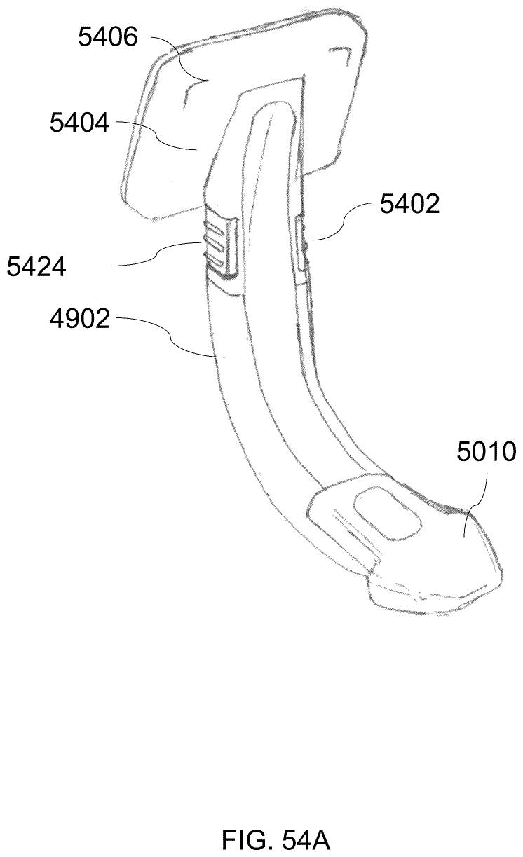

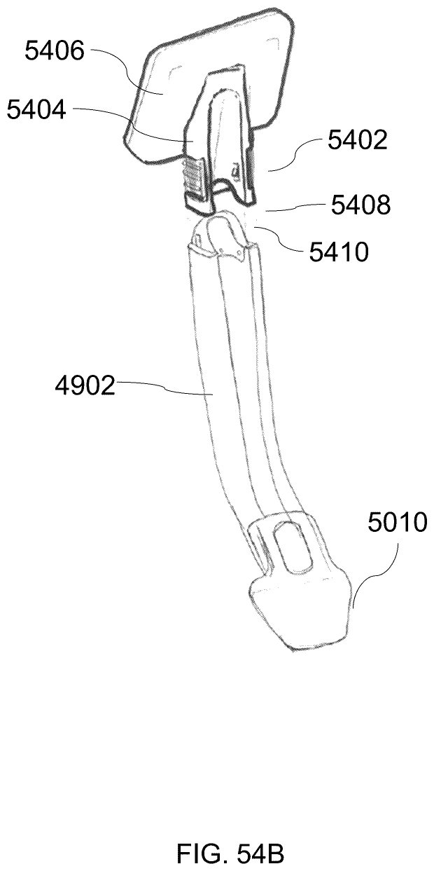

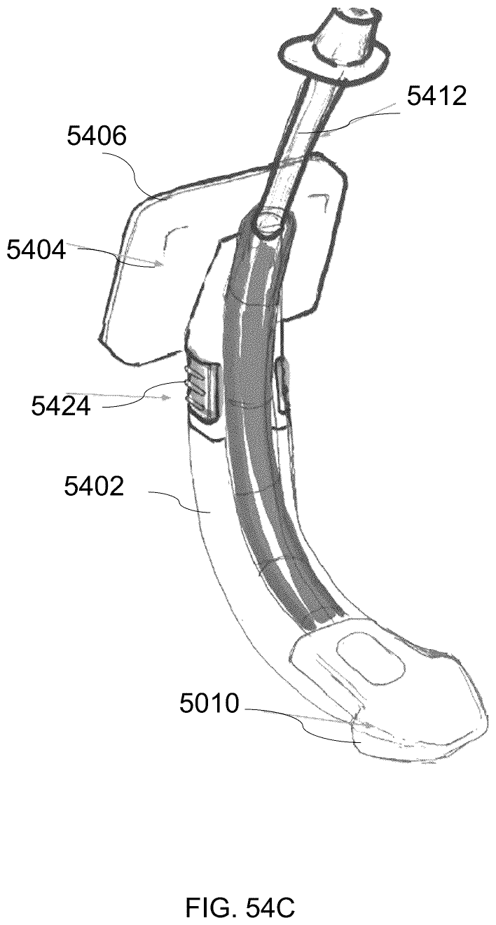

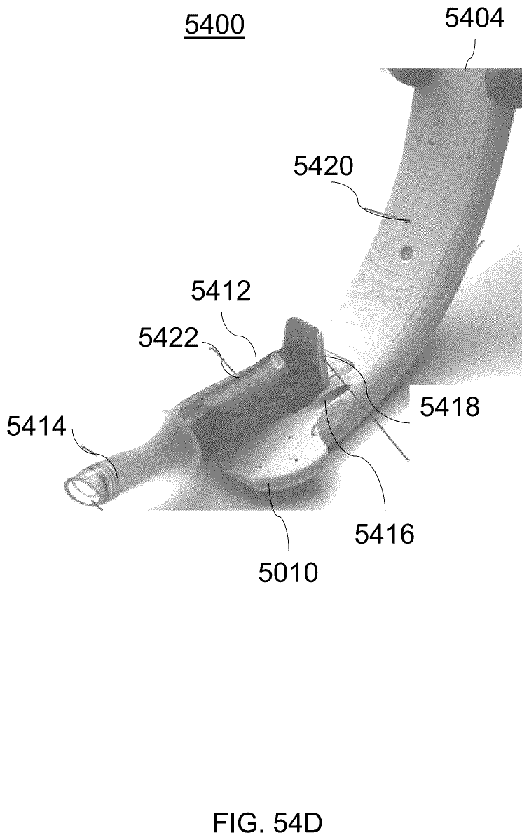

As discussed above, the device may benefit from some form of visualization. FIGS. 54A-54D show potential embodiments. In some exemplary embodiments, the additional features may be including for lighting and image collection capabilities properly located in the TAM to produce an optimal view. Possible features can include removability of the lighting and image collection subcomponents, and independent articulation of them via pull tendons (with specific bending regions).

FIGS. 54A-54C show a version of this concept including a removable, couplable display from the introducer and connecting through an interface. The tracheal intubation device can comprise a removable display or video monitor and a connector configured to releasably interface the video monitor with an introducer including a TAM and an interface having an ETT egress. FIGS. 54A-54C show rear perspective views of display holder 5404, display 5406, connector 5402, introducer 4902, ETT egress 5408, interface 5410, ETT 5412, controls 5424, and TAM 5010. The ETT egress 5408 can comprise a channel for securing an optical fiber, camera, or other visualization device. The display can be a video monitor. The video monitor can include a battery, image processing hardware and software, recording capabilities, and external interfaces and other components necessary for recording and displaying video. The controls 5424 can comprise controls for facilitating coupling and/or release of the display and the introducer. The controls 5424 can comprise controls for the display itself.

FIG. 54D shows an exemplary embodiment of an introducer 5400 including an integrated video monitor and removable endotracheal tube subassembly. FIG. 54D shows unibody introducer comprising removable handle 5420, ETT 5412, airway tube 5422, cuff 5414, TAM 5010, visualization port 5416, and epiglottic elevating element 5418. In an example, the display holder 5404 may secure to the proximal end of the introducer 5400. According to an exemplary embodiment, a camera may be integrated into the ETT subassembly, in manners previously described above. In each of these cases, any and each component may be reusable or disposable.

### Terms and Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein may not necessarily be construed to represent a substantial difference over what is generally understood in the art.

Throughout this application, various embodiments may be presented in a range format. It may be understood that the description in range format is merely for convenience and brevity and may not be construed as an inflexible limitation on the scope of the disclosure. Accordingly, the description of a range may be considered to have specifically disclosed all the possible subranges as well as individual numerical values within that range. For example, description of a range such as from 1 to 6 may be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers, and also represent an amount close to the stated amount that still performs a desired function or achieves a desired result. The term “about” or “approximately” may mean within an acceptable error range for the particular value, which will depend in part on how the value is measured or determined, e.g., the limitations of the measurement system. For example, the terms “approximately”, “about”, and “substantially” may refer to an amount that is within less than 10% of, within less than 5% of, within less than 1% of, within less than 0.1% of, and within less than 0.01% of the stated amount. For example, “about” may mean within 1 or more than 1 standard deviation, per the practice in the art. Alternatively, “about” may mean a range of up to 20%, up to 10%, up to 5%, or up to 1% of a given value. As used herein, the term “about” a number refers to that number plus or minus 10% of that number. The term “about” a range refers to that range minus 10% of its lowest value and plus 10% of its greatest value. Where particular values are described in the application and claims, unless otherwise stated the term “about” meaning within an acceptable error range for the particular value may be assumed.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The section headings used herein are for organizational purposes only and are not to be construed as limiting the subject matter described.

While various embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It may be understood that various alternatives to the embodiments of the disclosure described herein may be employed in practicing the disclosure. It is therefore contemplated that the disclosure shall also cover any such alternatives, modifications, variations, or equivalents. It is intended that the following claims define the scope of the disclosure and that methods and structures within the scope of these claims and their equivalents be covered thereby.

1.-91. (canceled)

92. A tracheal intubation system comprising:
a. an endotracheal tube (ETT) subassembly comprising a vine robot and an endotracheal tube;
b. an introducer configured to receive the vine robot; and
c. a tracheal access mechanism (TAM) coupled to the introducer, the TAM comprising:
i. a base having an egress for passing the ETT subassembly;
ii. a bridge extending from the base;
iii. a flexible datum coupled to the bridge; and
iv. a flap coupled to the bridge and configured to, in a collapsed state, cover at least a portion of a distal end of the egress and, in an actuated state, extend perpendicular to the surface coplanar to the bridge.

93. The tracheal intubation system of claim 92, wherein the introducer comprises a connected member and a pair of lateral rails configured to contact posterior tissue and to preserve a volume for a tubular channel between the lateral rails.

94. The tracheal intubation system of claim 93, further comprises an anterior surface connected between the pair of lateral rails, wherein the datum is connected to the pair of lateral rails and the anterior surface is configured to the support the datum.

95. The tracheal intubation system of claim 92, wherein the introducer comprises one or more channels configured to enable passage of one or more tools, cameras, or catheters.

96. The tracheal intubation system of claim 92, wherein the vine robot comprises a primary tube body comprising a sealed first end and a second end opposite the first end, wherein the first end is within a mesial portion of the primary tube body, and wherein the mesial portion comprises two or more stiffened portions, wherein adjacent stiffened portions are separated by a segment of the primary tube body.

97. The tracheal intubation system of claim 96, wherein the two or more stiffened portions are arranged from the first end to the second end and are radially aligned and arrayed about the primary tube body.

98. The tracheal intubation system of claim 97, wherein the two or more stiffened portions each comprise a stop coupled to the primary tube body, wherein the each stop of the two or more stiffened portions are interconnected by a tendon with a constant or actuatable length.

99. The tracheal intubation system of claim 96, wherein the primary tube body extends from the first end, through a cavity of an annular chassis, around an outer surface of the annular chassis and to the mesial portion of the tubular channel, wherein the annular chassis comprises one or more sensors.

100. The tracheal intubation system of claim 99, wherein the one or more sensors comprise one or more cameras, orientation sensors, temperature sensors, and pressure sensors.

101. The tracheal intubation system of claim 92, wherein the distal end of the tubular channel and a proximal end of the tubular channel comprise features to securely receive and release the vine robot.

102. The tracheal intubation system of claim 92, wherein the TAM is shaped and sized to lift an epiglottis of a patient anteriorly when the vine robot is fully actuated.

103. The tracheal intubation system of claim 92, wherein the vine robot is shaped and sized to extend through the patient's larynx when fully actuated.

104. The tracheal intubation system of claim 92, wherein the introducer comprises a directionally-compliant portion along a length of the introducer configured to bend easily when in contact on a posterior surface and to be rigid when in contact on an anterior surface.

105. The tracheal intubation system of claim 92, wherein the flexible datum comprises an upper esophageal sphincter (UES) self-centering width and molar avoidance geometry.

106. The tracheal intubation system of claim 92, further comprising one or more fiber optic cables, camera sensors, or lighting components.

107. The tracheal intubation system of claim 106, wherein the one or more fiber optic cables, camera sensors, or lighting components are electrically coupled to a display for visualizing a field of view from the TAM.

108. A method of intubating a patient comprising:
inserting the tracheal intubation system of claim 92 into a mouth of the patient to a hypopharynx of the patient;
advancing the vine robot distally;
disconnecting the TAM from the vine robot; and
removing the TAM and the introducer from the patient's mouth while retaining the vine robot within the patient.

109. The method of claim 108, further comprising lifting an epiglottis of the patient anteriorly, wherein lifting the epiglottitis anteriorly facilitates directing of the vine robot toward a trachea of the patient when the vine robot is actuated.

110. The method of claim 108, wherein the vine robot is advanced distally under visualization by (i) fiber optic cables or camera sensors, and (ii) displays associated with the tracheal intubation system.
